(19) United States
(12) Patent Application Publication
(10) Pub. No.: US 2003/0065305 A1
(43) Pub. Date: Apr. 3, 2003

(54) Method for stabilizing flux and decreasing lag-time during iontophoresis

(76) Inventors: William I. Higuchi, Salt Lake City, UT (US); David J. Miller, Bountiful, UT (US); S. Kevin Li, Salt Lake City, UT (US); Matthew S. Hastings, Sandy, UT (US)

Correspondence Address: REED & EBERLE LLP, 800 MENLO AVENUE, SUITE 210, MENLO PARK, CA, 94025, US

(21) Appl. No.: 10/226,622

(22) Filed: Aug. 21, 2002

Related U.S. Application Data
(63) Continuation-in-part of application No. 09/911,594, filed on Jul. 23, 2001.

(51) Int. Cl. A61N 1/30

(52) U.S. Cl. 604/501; 604/20

(57) ABSTRACT

An iontophoretic method for transporting compounds of interest across a body tissue is provided. The method can be used to extract analytes or deliver drugs. The method utilizes a polyelectrolyte and provides for the maintenance of a substantially constant flux across a localized region of the tissue through which transport occurs, thereby allowing a compound of interest to be transported across the tissue in a controlled and predictable manner. In addition, the presence of the polyclectrolyte reduces the lag-time of molecular transport through the body tissue.

### CROSS REFERENCE To RELATED APPLICATIONS

This is a continuation-in-part of U.S. patent application Ser. No. 09/911,594, filed Jul. 23, 2001, which is incorporated by reference herein in its entirety.

### FIELD OF THE INVENTION

This invention relates generally to the use of iontophoresis and, more specifically, to a novel method for stabilizing the flux of charged and uncharged compounds through a body surface. This invention finds utility in any instance where a compound is removed from the body via iontophoresis, such as glucose monitoring, phenylalanine monitoring, therapeutic drug monitoring, sobriety monitoring, fertility monitoring, monitoring for illicit drug use, noninvasive pharmacokinetic or toxicokinetic monitoring, and monitoring of any other body component, endogenous or introduced, that is a marker of health or disease. This invention also finds utility in any instance where a compound is delivered to the body via iontophoresis to achieve a therapeutic effect. In addition, this invention also reduces the lag time of iontophoretic systems.

### BACKGROUND OF THE INVENTION

The transport of various compounds such as endogenous species, metabolites, drugs and nutrients across body surfaces such as the skin or mucosal tissue is primarily a function of three factors: tissue permeability; the presence, absence, and magnitude of a driving force; and the size of the area through which transport occurs. Body tissues such as skin, mucosal tissue, and ocular tissue are generally not sufficiently permeable to allow therapeutic concentrations of drug molecules following passive transport therethrough. For example, the stratum corneum of the skin presents the primary barrier to absorption of topical compositions or transdermally administered drugs. The stratum corneum is a thin layer of dense, highly keratinized cells approximately 10-15 microns thick over most of the body. It is believed to be the high degree of keratinization within these cells as well as their dense packing and intercellular lipids that creates, in most cases, a substantially impermeable barrier to drug penetration. Similar barriers exist for other body surfaces. As with other tissues in the body, the rate of permeation of many drugs through body surfaces is extremely low without the use of some means to enhance their permeability.

Iontophoresis is one approach that can be utilized to transport compounds of interest across a body surface through the application of a low level electrical current. In practice, iontophoretic devices utilize at least two electrodes, each positioned so as to be in intimate electrical contact with some area of the body surface, i.e., skin, eye or mucosal tissue. The device is also equipped with a reservoir connected to one of the electrodes to provide a source of the molecules or ions to be delivered or a holding chamber for molecules or ions that are extracted. An electrical circuit is formed by connection of these electrodes to a source of electrical energy, e.g., a battery and circuitry capable of controlling the amount of current passing through the device and body surface.

For drug delivery, one electrode, referred to as the “active” or “donor” electrode, is the electrode from which the drug is delivered into the body. The active donor can also serve to collect analytes from the body. The other electrode, referred to as the “counter” or “return” electrode, serves to close the electrical circuit through the body. This second electrode may also have delivery or sensing capabilities built in. When the ionic substance to be driven into the body is positively charged, then the positive electrode (the anode) acts as the active electrode and the negative electrode (the cathode) serves as the counter electrode, thereby completing the circuit. Conversely, if the ionic substance to be delivered is negatively charged, then the cathode is the active electrode and the anode is the counter electrode.

Another particular type of iontophoresis is referred to as “reverse” iontophoresis, and relates to the withdrawal of compounds from the body by transport across a body surface. Reverse iontophoresis can be used to withdraw both charged and uncharged compounds from the body, often referred to as analyte extraction. In reverse osmosis, the electrode receiving the analyte is the “receiver” or “sensing” electrode, while the second electrode is the “indifferent” or “return” electrode. A reservoir connected to one of the electrodes serves as a receiving chamber for the analyte being extracted. Due to the direction of electroosmotic force or direct electrostatic attraction, most analytes are collected at the cathode. However for strongly anionic compounds, the anode may serve as the analyte receiving chamber.

Reverse iontophoresis is a noninvasive method and therefore has wide utility in analytical applications. Reverse iontophoresis can be used to extract glucose from a patient's body, the concentration of which is then used to determine blood glucose levels in the patient. This analytical process can be readily adapted to provide a thorough picture of a patient's blood glucose profile on a real-time basis. Reverse iontophoresis can also be used to extract disease markers. For example, phenylalanine can be extracted from the body of a patient suffering from phenylketonuria in order to detect whether phenylalanine is accumulating in the patient's blood, or it can be extracted as part of a screening method to identify patients having elevated phenylalanine plasma levels. Another use of reverse iontophoresis relates to the extraction of agents having a narrow therapeutic window, such as aminoglycoside antibiotics, antiepileptic agents, cardiac glycosides, and anticoagulants, so that drug levels in the blood can be monitored and the drug dosage adjusted as necessary. Other uses of reverse iontophoresis include detection of toxic substances, detection of illicit drugs, and toxicokinetic or pharmacokinetic monitoring.

There are three aspects to iontophoretic transport: direct electric field effect, electroosmosis and electroporation, the latter involving transport within aqueous pores that exist in membrane structures or are created therein by application of an electrical current. Much research has focused on understanding and optimizing electroosmosis, a process that typically depends upon sodium ion flow into the cathode from the body. Electroosmotic flow is created by an electrical volume force that results from mobile ions, located within the pores, acting upon the solvent. State of the art reverse iontophoretic devices contain a high concentration of these small, highly mobile ions in the receiver chamber. As current is applied, these ions enter the transport pathways and create an inward driving force that impedes the outward extraction convection force. In addition, the presence of small, mobile ions seems to increase the lag time and provides for less steady-state stability in molecular transport.

In order to optimize reverse iontophoretic methods and devices, it is important to develop reproducible extraction processes and also to increase the rate of analyte extraction. Various methods have been explored to increase the rate of electroosmotic extraction. Santi, et al., J. Controlled Release 38:159-165 (1996) showed that the rate of electroosmotic flux can be increased by lowering the ionic concentration of electrolyte in both the anode and cathode chambers. However, the use of very low ionic strength solutions in the extraction compartment is disadvantageous in a number of respects. For example, a higher voltage is required, thereby increasing the potential for skin irritation. In addition, an inadequate number of ions is provided to support the necessary electrochemical reactions at the electrode surfaces. Lastly, they achieved only about two-fold maximum improvement in electroosmotic flux into the cathode chamber and even less into the anode chamber.

The problem with most iontophoretic devices, including constant current and constant conductance systems, is the substantial amount of energy required to achieve and maintain a target state of electroporation and transport rate. Iontophoresis can cause irritation, sensitization and pain in some patients, and the degree of irritation, sensitization and/or pain is, as a general rule, directly proportional to the applied current or voltage. The effects of the electrical current on sensitization have been investigated, resulting in attempts to develop iontophoretic devices and methods that are capable of maintaining the electrical current and/or potential at a comfortable level. For example, Haynes et al., U.S. Pat. No. 5,246,418 describes a method of reducing irritation during iontophoresis using a feedback circuit, which, during iontophoretic transport, enables control over the applied current and voltage.

Therefore, in spite of the advances in the art, there continues to be a need for improved iontophoretic methods that allow for increased levels of permeant transport in electroosmosis, while minimizing irritation, sensitization and pain. The present invention addresses those needs by replacing the mobile co-ions (which are capable of easily entering the pores from the receiver compartment of an iontophoretic device) with large conductive polyelectrolytes. Thus, the invention significantly improves the amount of compound extracted, improves device performance, decreases energy requirements, increases battery life, reduces the potential for irritation, and improves accuracy, reproducibility, and precision.

### SUMMARY OF THE INVENTION

One aspect of the invention relates to a method of decreasing flux variability in an iontophoretic device used to transport a compound of interest through a localized region of a patient's body tissue, comprising: (a) applying a current to the localized region of body tissue at a level sufficient to effect iontophoretic transport of the compound of interest therethrough; (b) either prior to, during, or both prior to and during application of the current, applying to the localized region of body tissue an amount of at least one polyelectrolyte effective to stabilize the rate of flux of the compound of interest through the localized region of body tissue.

Another aspect of the invention pertains to a method of decreasing lag time of the iontophoretic transport of a compound of interest through a localized region of a patient's body tissue, comprising: (a) applying a current to the localized region of body tissue at a level sufficient to effect iontophoretic transport of the compound of interest therethrough; (b) either prior to, during, or both prior to and during application of the current, applying to the localized region of body tissue an amount of at least one polyelectrolyte effective to decrease the time needed to achieve steady state transport of the compound of interest through the localized region of body tissue.

### DETAILED DESCRIPTION OF THE INVENTION

The present invention relates to an iontophoretic method for transporting a compound of interest across a body tissue utilizing current in conjunction with a polyelectrolyte. The method can be used to deliver or extract a number of different compounds, such as endogenous substances located within the body, pharmacologically active agents, markers of disease, and the like. During iontophoretic transport, the polyelectrolyte stabilizes the flux of the compound of interest, thereby allowing compounds to be transported across the tissue in a controlled and predictable manner. The polyelectrolyte also serves to significantly reduce the lag-time of the iontophoretic system. The method has utility in a wide range of applications, e.g., in therapeutic treatments, in detoxification methods, in pain management, in metabolite or therapeutic agent monitoring, and dermatological treatments. The method of the invention can also be utilized in diagnostic applications, e.g., to detect the presence of a disease marker.

In many iontophoretic systems, the net solvent convective flow and resulting compound movement is in the direction of anode to cathode. Although most uses of electroosmosis utilize net convection in the direction of anode to cathode, this invention is not limited to transport in the direction of anode to cathode. A polycationic substance will increase permeant transport in the direction of cathode to anode. Unexpected advantages of reversing the traditional electroosmotic flow could include a possible decrease in irritation, decrease in electrical requirement, or increase in the amount of permeant extracted through the skin per unit time and increased precision, reproducibility, and accuracy.

Lastly, the use of polyelectrolytes may increase drug delivery via electroosmotic or non-electroosmotic routes from both the anode and cathode, when the drug is positively charged.

I. Definitions

Before describing the present invention in detail, it is to be understood that this invention is not limited to specific drug delivery systems, reverse iontophoresis extraction systems, iontophoretic device structures, polyelectrolytes, carriers, or the like, as such may vary. The definitions set forth apply only to the terms as they are used in this patent and may not be applicable to the same terms as used elsewhere, for example in scientific literature or other patents or applications including other applications by these inventors or assigned to common owners. The following description of the preferred embodiments and examples are provided by way of explanation and illustration only and is not intended to be limiting. As such, they are not to be viewed as limiting the scope of the invention as defined by the claims. Additionally, when examples are given, they are intended to be exemplary only and not to be restrictive.

It must be noted that, as used in this specification and the appended claims, the singular forms “a”, “an” and “the” include plural referents unless the context clearly dictates otherwise. Thus, for example, reference to “a polyelectrolyte” includes a mixture of two or more such compounds, as well as a single polyelectrolyte, reference to “an analyte” includes one or more analytes, and the like.

In describing and claiming the present invention, the following terminology will be used in accordance with the definitions set out below.

The terms “iontophoresis” and “iontophoretic” are used herein to refer to the delivery of pharmaceutically active agents or the extraction of charged and uncharged compounds from the human body through a body surface by means of an applied electromotive force to an agent-containing reservoir. The terms “iontophoresis” and “iontophoretic” are also meant to refer to “reverse iontophoresis,” “reverse iontophoretic,” “electroosmosis,” and “iontohydrokinesis” or “iontohydrokinetic.” The terms “reverse iontophoresis,” “reverse iontophoretic,” and “analyte extraction” are used to refer to the collection of analytes from the body to an analyte-collecting reservoir by means of an applied electromotive force.

The term “polyelectrolyte” is used to describe any molecule, ion or particle, organic or inorganic, that is charged (negatively charged, positively charged, or zwitterionic), or that is capable of being rendered charged. Polyelectrolytes have at least one, and preferably two or more charged groups and associated counter-ions. The term “polyelectrolyte” also includes a mixture or mixtures of different polyelectrolytes or similar polyelectrolytes with different molecular weight distributions. The “polyelectrolyte” may be a single molecule or an aggregate of molecules, such as micelles and liposomes. If the polyelectrolyte is particulate, i.e., comprised of a plurality of molecular aggregates, the particles can be porous or nonporous, and may be, for example, macromolecular structures such as micelles (cationic or anionic) or liposomes (cationic or anionic). Polyelectrolytes may be in solution form or present in a suspension, dispersion, or colloidal system.

“Co-ion” is used to define an ion that is transported in the same direction as the electrical current (e.g., away from the electrode with similar polarity to its ionic charge, i.e., anions are co-ions at the cathode and vice versa). Other terms that are synonymous with “co-ion” are “background ion,” “background electrolyte,” and “excipient ion. “Counter-ion” is used to define an ion that is transported contrary to the electrical current (e.g., towards the electrode with similar polarity to its ionic charge, i.e., anions are counter-ions at the anode and vice versa).

The terms “current” and “electrical current,” when used to refer to the conductance of electricity by movement of charged particles, are not limited to “direct electrical current,” “direct current,” or “constant current.” The terms “current” or “electrical current” should also be interpreted to include “alternating current,” “alternating electrical current,” “alternating current with direct current offset,” “pulsed alternating current,” and “pulsed direct current.”

During iontophoresis, certain modifications or alterations of the body surface occur, for example, changes in permeability, due to mechanisms such as the formation of transiently existing pores, also referred to as “electroporation.” Any electrically assisted transport of species enhanced by modifications or alterations to the body surface (e.g., formation of pores in the skin and “electroporation”) are also included in the term “electrotransport” as used herein. Thus, as used herein, the terms “electrotransport,” “iontophoresis,” and “iontophoretic,” further refer to the transport of permeants by the application of an electric field regardless of the mechanisms.

The term “pore” is used to describe any transport pathway through the tissue, whether endogenous to the tissue or formed by electroporation.

As used herein, a “body tissue” refers to an aggregation of similar cells and/or cell components united in performance of a particular function. The tissue can be part of a living organism, a section excised from a living organism, or artificial. Typically, however, the body tissue will be the body surface of a patient, i.e., skin, mucosal tissue (including the interior surface of body cavities that have a mucosal lining), ocular tissue (e.g. conjunctiva, sclera and cornea), etc. The terms “skin” and “mucosal tissue” are used interchangeably. Typically the patient will be human, however, the invention also finds utility on small mammals, birds, farm and other domesticated animals, as well as animals found in the wild and in zoological parks.

A “localized region” of a tissue refers to the area or section of a body tissue through which a compound of interest is transported. Thus, a localized region of a body surface refers to an area of skin or mucosal tissue through which an active agent is delivered or an analyte is extracted.

The term “transport,” as in the “transport” of a compound of interest across a body tissue, refers to passage of the compound in either direction, i.e., the compound may be delivered to the patient from an external source, across the skin or mucosal tissue, or it may be extracted from beneath the patient's body surface to the exterior of the body, as in analyte extraction.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. The term “treatment” is also used to refer to the extraction of a substance through a body tissue for the purpose of quantitative or qualitative analysis.

The terms “drug,” “active agent,” and “pharmacologically active agent” are used interchangeably herein to refer to any chemical compound, complex or composition, charged or uncharged, that is suitable for topical, transdermal, ocular, or transmucosal administration and that has a beneficial biological effect, preferably a therapeutic effect in the treatment of a disease or abnormal physiological condition, although the effect may also be prophylactic in nature. The terms also encompass agents that are administered for nutritive or diagnostic purposes, e.g., nutrients, dietary supplements and imaging agents. The terms also encompass pharmaceutically acceptable, pharmacologically active derivatives of those active agents specifically mentioned herein, including, but not limited to, salts, esters, amides, prodrugs, active metabolites, analogs, and the like. When the terms “active agent,” “pharmacologically active agent” and “drug” are used, then, or when a particular active agent is specifically identified, it is to be understood that applicants intend to include the active agent per se as well as pharmaceutically acceptable, pharmacologically active salts, esters, amides, prodrugs, conjugates, metabolites, analogs, etc.

The term “analyte” is used to refer to a compound, molecule or ion to be iontophoretically extracted from beneath a localized region of a patient's body tissue. When particular types of analytes are mentioned, it is to be understood that salts, esters, amides, analogs, conjugates, metabolites and other derivatives are included unless otherwise indicated.

The term “compound of interest” is used collectively to refer to drugs and analytes, and included charged and uncharged species, ions, molecules, chemical compounds and compositions.

The terms “optional” and “optionally” mean that the subsequently described circumstance may or may not occur, so that the description includes instances where the circumstance occurs and instances where it does not. For example, recitation of an “optional” DC offset encompasses an iontophoretic process without a DC offset as well as an iontophoretic process with a DC offset. It must be noted that the invention is not limited to constant current or direct current iontophoretic methods, but encompasses alternating current iontophoresis as well as AC iontophoresis with a direct current offset.

II. Methods of Stabilizing Flux and Decreasing Lag-Time During Iontophoretic Transport

The methods of the invention serve to stabilize flux during iontophoresis. Two common problems with state of the art systems are flux drift and the long time needed to achieve steady state. This flux drift is, at least in part, responsible for presently available commercial devices' need for frequent re-calibration during extraction and imprecision during drug delivery. The need for frequent re-calibration is problematic with extraction devices since the devices typically require a two or more hour warm up period before calibration can take place. In addition, the calibration procedure for analyte extraction is invasive, as it requires taking a blood sample by traditional invasive means. It is expected that the methods described herein will require less frequent calibrations, and thus will provide for a longer operational period between calibrations. It is expected that the methods of the invention will provide for a stabilized flux such that the systems can operate for up to 7 days before re-calibration is needed. Fewer re-calibrations also provides for fewer warm-up periods, the advantage of which is that it provides for increased patient convenience, as well as expanding the utility of the device.

Flux drift and lag time are equally problematic for drug delivery devices. During the non-steady state portion of the delivery cycle, the flux will continually change. In addition, as the flux drifts during the course of treatment, the amount of drug delivered will continually change, making the prediction of the precise amount of medicament delivered over a period of time imprecise. This imprecision could lead to delivery of subtherapeutic amounts of drug to some patients and toxic amounts to others. Further, as the treatment continues, a patient that received subtherapeutic amounts in the treatment's beginning, may receive toxic amounts towards the treatment's end, and vice versa. Thus, the lag-time and flux drift that are problematic with analyte extraction, present an equal challenge to drug delivery.

Accordingly, one embodiment of the invention is a method of decreasing flux variability in an iontophoretic device used to transport a compound of interest through a localized region of a patient's body tissue, comprising: (a) applying a current to the localized region of body tissue at a level sufficient to effect iontophoretic transport of the compound of interest therethrough; (b) either prior to, during, or both prior to and during application of the current, applying to the localized region of body tissue an amount of at least one polyelectrolyte effective to stabilize the rate of flux of the compound of interest through the localized region of body tissue.

The methods of the invention provide for the maintenance of a substantially constant flux across a localized region of the tissue through which transport occurs, thereby allowing a compound of interest to be transported across the tissue in a controlled and predictable manner. In a preferred embodiment, this method of the invention provides for at least a 25% decrease in variability in the flux provided, as compared to the variability observed in the absence of the polyelectrolyte. More preferably, the method provides for at least a 50% decrease in flux variability, and most preferably, the method provides for at least a 75% decrease in flux variability.

In one embodiment of the invention, the decreased variability is expressed as decreased intrasubject variability. In another embodiment, the decreased variability is expressed as decreased intersubject variability.

The method involves applying at least one polyelectrolyte to the to the localized region of body tissue. Suitable polyelectrolytes are described below. The polyelectrolyte can be placed in direct contact with the tissue surface. Direct contact can be achieved by incorporating the polyelectrolyte into the membrane-contacting layer, for example in a reservoir or adhesive layer affixed to the tissue. Alternately, the polyclectrolyte can be placed in indirect contact with the tissue, for example located in a reservoir that is separated from the tissue by a membrane or adhesive layer.

As noted above, the method of the invention involves operating the iontophoretic device to apply a current to a localized region of body tissue. Typically, this localized region of body tissue will have an area within the range of about 0.1-100 cm². In a preferred embodiment, this region has an area within the range of about 0.5-30 cm², more preferably 1-20 cm².

In one embodiment, the current is alternating current (AC), which generally refers to an electric signal (e.g., current or voltage) that reverses direction periodically. The AC signal is typically adjusted to maintain a substantially constant state of electroporation in the localized region of tissue throughout the time period in which the compound of interest is transported. The electrical state that is maintained by the AC signal is electrical conductance or electrical resistance, generally the latter. The AC signal applied to the tissue can have essentially any waveform. The waveform can be symmetric or asymmetric, thus including square, sinusoidal, saw-tooth, triangular and trapezoidal shapes, for example. Typically, the alternating current is applied to the localized region of the body tissue for a time period within the range of approximately 2 minutes to greater than 72 hours. In one embodiment, the time period is within the range of approximately 12-72 hours, and preferably within the range of approximately 12-48 hours. Typically, the AC is applied at a voltage level within the range of about 1-75 V. In one embodiment, the voltage level is within the range of about 1-45 V, preferably within the range of about 1-30 V. Typically, the frequency of the AC signal tends to be at least about 1 Hz, although in other instances the frequency is within the range of about 1 Hz to about 1 kHz, about 1 kHz to about 10 kHz, or about 10 kHz to about 30 kHz.

In another embodiment, the invention can also use direct current (DC). Typically, the DC is applied to the localized region of the body tissue for a time period within the range of approximately 2 minutes to greater than 72 hours. In one embodiment, the time period is within the range of approximately 12-72 hours, and preferably within the range of approximately 12-48 hours. Typically, the DC is applied at level within the range of about 0.01-0.5 mA/cm². In one embodiment, the voltage level applied is within the range of about 0.1-0.5 mA/cm², preferably within the range of about 0.1-0.3 mA/cm².

Another embodiment of the invention is a method of decreasing lag time of the iontophoretic transport of a compound of interest through a localized region of a patient's body tissue, comprising: (a) applying a current to the localized region of body tissue at a level sufficient to effect iontophoretic transport of the compound of interest therethrough; (b) either prior to, during, or both prior to and during application of the current, applying to the localized region of body tissue an amount of at least one polyclectrolyte effective to decrease the time needed to achieve steady state transport of the compound of interest through the localized region of body tissue.

In a preferred embodiment, this invention provides for at least a 20% reduction in lag-time compared to the lag-time in the absence of the polyelectrolyte. More preferably, the method provides for at least a 40% reduction in lag-time, even more preferably at least a 60% reduction in lag-time. A major shortcoming with state of the art iontophoretic systems is that they possess long lag-times, often up to three hours or more. With a decreased lag-time, the methods of the invention can also be used to significantly decrease the warm-up that is needed with most iontophoretic devices.

The aforementioned methods of the invention are designed to accomplish transport of a compound of interest across a body tissue and more specifically across a localized region of body tissue. As used herein a “tissue” is defined to mean an aggregation of similar cells and/or cell components united in performance of a particular function. The tissue can be part of a living organism, a section excised from a living organism, or artificial. An artificial tissue is one in which an aggregation of cells are grown to function similar to a tissue in a living organism. The aggregated cells, however, are not obtained from a host (i.e., a living organism). Artificial tissues can be grown in vivo or in vitro. Human skin, for instance, can be cultured in vitro to obtain an aggregation of cells, of monolayer thickness or greater, that can function as a skin tissue in culture or once grafted onto a living host. Certain types of artificial tissues that can be utilized with certain methods of the invention are discussed, for example, in U.S. Pat. No. 4,458,678 to Yannas et al., U.S. Pat. No. 4,485,096 to Bell, and U.S. Pat. No. 4,304,866 to Green at al.

Certain methods are performed with human or animal tissue. Thus, the invention may be used in various clinical applications for human patients, as well as veterinary applications. In the latter context, the invention may be used with any animal having body tissues in which pores can be generated via the application of an electrical signal. Hence, some methods can be performed, for example, with domestic animals such as dogs and cats; farm animals such as horses, cows, sheep and pigs; exotic animals; birds; reptiles; and amphibians, or tissues from these animals. Still other methods are performed with plants or plant cell cultures.

It is an added advantage of the methods of the invention that an enhanced flux is obtained. The use of high molecular weight, charged polyelectrolyte polymers to provide an electrically conducting medium in the receiving and/or donor electrode may also result in a 50% to a 50-fold or more improvement in the iontophoretic transport of compounds of interest. In a preferred embodiment, at least 50%, more preferably at least a 100%, and even more preferably at least a 200% enhanced flux of the compound of interest is obtained, as compared to the flux in the absence of the polyelectrolyte.

According, another embodiment of the invention is a method of increasing the flux of a compound of interest during ionic transport conducted on a localized region of a patient's body tissue, comprising: (a) applying a current to the localized region of body tissue at a level sufficient to effect iontophoretic transport of the compound of interest therethrough; (b) either prior to, during, or both prior to and during application of the current, applying to the localized region of body tissue an amount of at least one polyelectrolyte effective to increase the flux of the compound of interest through the localized region of body tissue.

III. Polyelectrolyte

Polyelectrolytes are polymers having ions or ionizable groups. In one embodiment, the polyelectrolyte is selected so as to have a molecular weight of about 200 Da or greater. In another embodiment, the polyelectrolyte has a molecular weight in the range of 200 to 1000 Da. In yet another embodiment, the polyelectrolyte has a molecular weight of greater than 1000 Da. The polyelectrolyte may also have a molecular weight in the range of 1000 to 10, 000 Da

In one embodiment, the polyelectrolyte will have a size sufficient to hinder its entrance into the transport pathways. It is expected that compounds having a minimal size of 1000 Da are poorly transported through body tissue, if at all, even under the influence of an electrical current. However, in another embodiment, the polyelectrolytes enter the transport pathways and affect their charge characteristics, thereby enhancing transport.

In one embodiment of the invention, a membrane is positioned between the polyelectrolyte and the body tissue so as to sequester transport of the polyelectrolyte through the transport pathways. Such a membrane would typically have a pore size that would allow the analyte or drug being transported to pass through relatively unimpeded.

The polyelectrolyte can be selected from the group consisting of cationic polyelectrolytes, anionic polyelectrolytes, nonionic polyclectrolytes, amphoteric polyelectrolytes, and mixtures thereof. In a preferred embodiment, the polyelectrolyte is a compound having at least one ionic group.

Cationic polyelectrolytes commonly contain quaternary ammonium; primary, secondary, or tertiary amines charged at the reservoir solution pH; heterocyclic compounds charged at reservoir solution pH; sulfonium; or phosphonium groups. Anionic polyelectrolytes typically contain one or more carboxylate, sulfonate and phosphate groups. In addition, polyclectrolytes having characteristics of more than one of these categories may also be used in the methods of the invention. For example, partial hydrolysis of a compound such as polyacrylamide produces an amphoteric polyelectrolyte that has both amide (nonionic) and carboxylic acid (anionic) groups. Accordingly, the polyelectrolyte can comprise one or more ionic groups selected from the group consisting of quaternary ammonium, sulfonium, phosphonium, carboxylates, sulfonates and phosphates.

Exemplary backbone structures for such polyelectrolyte compounds include, by way of illustration and not limitation, acrylamides, addition polymers (e.g., polystyrenes), oligosaccharides and polysaccharides (e.g., agaroses, dextrans, celulloses), polyamines and polycarboxylic acid salts, polyethylenes, polyimines, polystyrenes, and mixtures thereof.

Exemplary cationic polyelectrolytes include, by way of illustration and not limitation, the following compounds:

addition polymers such as polyvinyl alcohol and other polyvinyl compounds such as poly(vinyl 4-alkylpyridinium), poly(vinylbenzyltrimethyl ammonium, and polyvinylimine;

aminated styrenes;

cholestyramine;

polyimines such as polyethylenimine;

aminated polysaccharides, particularly cross-linked polysaccharides such as dextrans (e.g., dextran carbonates and DEAE dextran);

and mixtures thereof.

Exemplary anionic polyelectrolytes include, by way of illustration and not limitation, the following compounds:

acrylamides such as acrylamideo methyl propane sulfonates (poly-AMPS), poly(N-tris[hydroxymethyl]methyl methacrylamide and other anionic copolymers of acrylamide;

alginate and alginic acid;

addition polymers such as homopolymers and copolymers of derivatives of acrylate and methacrylate [e.g., hydroxyl ethyl methacrylates (poly-HEMA), poly (2-DEAE methacrylate) phosphate, and poly(ethyl acrylate-co-maleic anhydride-co-vinyl acetate) sodium; including salts thereof such as sodium polyacrylates]; and polystyrenes [e.g., polystyrene sulfonate, sodium polystyrene sulfonate, sodium polystyrene sodium sulfonate (“NaPSS”), and poly (maleic anhydride-co-styrene) 2-butoxyethyl ester, ammonium salt]; as well as esters and amides thereof having free hydroxyl functionalities;

hyaluronate;

oligosaccaharides such as the anionically charged cyclodextrans (e.g., sulfobutyl ether β-cyclodextrans);

pectic acid;

polyacrylic acids (e.g., poly(acrylic acid-do-ethylene) sodium);

polysaccharides, particularly cross-linked polysaccharides such as dextrans (e.g., dextran sulfonates and heparin);

polystyrenesulfonic acids;

polyvinylphosphonic acids;

and mixtures thereof.

Exemplary nonionic polyelectrolytes include, by way of illustration and not limitation, polyacrylamide and polyvinyl alcohol.

As can be seen from above, many materials or polymeric backbones can be either anionic or cationic, depending upon the substituent groups. Accordingly, there are numerous other materials that are suitable for use as polyelectrolytes, either as is or by modification to include ionic groups. These include the following:

heparin and heparin derivatives;

liposomes, both anionic and cationic;

micelles, both anionic and cationic;

polyamines such as polyvinylpyridine;

polyethylenes including chlorosulfonated polyethylene, poly(4-t-butylphenol-co-ethylene oxide-co-formaldehyde) phosphate, polyethyleneaminosteramide ethyl sulfate, poly(ethylene-co-isobutyl acrylate-co-methacrylate) potassium, poly(ethylene-co-isobutyl acrylate-co-methacrylate) sodium, poly(ethylene-co-isobutyl acrylate-co-methacrylate) sodium zinc, poly (ethylene-co-isobutyl acrylate-co-methacrylate) zinc; poly(ethylene-co-methacrylic acid-co-vinyl acetate) potassium; polyethyleneimine, and poly(ethylene oxide-co-formaldehyde-co-4-nonylphenol) phosphate;

polysaccharides, including cross-linked polysaccharides such as agaroses, celluloses [e.g., benzoylated naphthoylated diethylaminoethyl (DEAE) cellulose, benzyl DEAE cellulose, triethylaminoethyl (TEAE) cellulose, carboxymethylcellulose, cellulose phosphate, DEAE cellulose, epichlorohydrin triethanolamine cellulose, oxycellulose, sulfoxyethyl cellulose and QAE cellulose], starch, and the like;

and mixtures thereof.

There are numerous polyelectrolytes within the aforementioned classes that are well suited for use in the invention and are commercially available from sources such as Sigma. For example the polyelectrolyte can be a polysaccharide such as the agaroses sold under the name Sepharose® (Pharmacia Biotech AB) such as CM Sepharose, DEAE Sepharose, Q Sepharose, and SP Sepharose; and the dextrans sold under the name Sephadex® (Pharmacia) such as CM Sephadex, DEAE Sephadex, SO Sephadex, QAE Sephadex.

Many of the aforementioned examples of polyelectrolyte materials are broadly classified as ion exchange materials, which are of particular interest for use in the invention. In general, ion exchange materials are highly ionic, covalently cross-linked, insoluble polyelectrolytes supplied as beads. Exemplary ion exchange materials include, by way of illustration and not limitation, polyacrylic acids, polyacrylic sulfonic acids, polyacrylic phosphoric acids and polyacrylic glycolic acids, polyvinyl amines, polystyrenes, poly epichlorohydrin/tetraethylenetriamines, and polymers having pendent amine groups including aromatic amines. The ion exchange resin can be a strongly acidic cation exchange resin (characterized by containing sulfonic acid groups or the corresponding salts); a weakly acidic cation exchange resin (characterized by containing carboxylic acid groups or the corresponding salts); a strongly basic anion exchange resin (characterized by containing quaternary ammonium groups), either those containing trialkyl ammonium chloride or hydroxide or those containing dialkyl 2-hydroxyethyl ammonium chloride or hydroxide; a weakly basic anion exchange resin (characterized by containing ammonium chloride or hydroxide), and a mixed bed resin. These ion exchange resins are sold under numerous tradenames such as Amberlite® and Amberjet® (both Rohm & Haas Company), Dowex® (Dow Chemical Co), Diaion® (Mitsubishi Kasei Corporation), Duolite® (Duolite International Inc.), Trisacryl® (Sepracor S.A. Corp.), and Toyopearl® (Toyo Soda Manufacturing Co., Ltd.).

Exemplary strongly acidic cation exchange resins include, by way of example and not limitation, Amberlite IRP-69, Amberlite IR-120 Plus, Amberlite IR-122, Amberlite IR-130C,

Exemplary weakly acidic cation exchange resins include, by way of example and not limitation, Amberlite CG-50, Toyopearl DEAE-650C, and Toyopearl DEAE 650-M.

Exemplary general anion exchange resins include, by way of example and not limitation, Amberlite IRA-458.

Exemplary strongly basic anion exchange resins include, by way of example and not limitation, Amberlite IRA-400, Amberlite IRA-402, Amberlite IRA-410, Amberlite IRA-420C, Amberlite IRA-743, Amberlite IRA-900, Amberjet 4400; Dowex 1X2-100, Dowex 1X2-200, Dowex 1X2-400, Dowex 1X4-50, Dowex 1X4-100, Dowex 1X4-200, Dowex 1X4-400, Dowex 1X8-50, Dowex 1X8-100, Dowex 1X8-200, Dowex 1X8-400, Dowex 2X8-100, Dowex 2X8-200, and Dowex 2X8-400.

Exemplary weakly basic anion exchange resins include, by way of example and not limitation, Amberlite IRA-67.

Exemplary mixed bed resins include, by way of example and not limitation, Amberlite IRN-150, Dowex MR-3, and Dowex MR-3C.

Other ion exchange resins within the aforementioned classes include:

Amberlite strongly acidic, Amberlite 200, Amberlite A 5836, Amberlite D 7416, Amberlite DP-1, Amberlite I 6641, Amberlite I 6766, Amberlite IRP-64, Amberlite IRP-88, Amberlite IR-1118H, Amberlite IRA-92, Amberlite IRA-95, Amberlite IRA-96, Amberlite IRC-50, and Amberlite MB-150;

Diaion Strongly acidic, Diaion 1-3501, Diaion 1-3513, Diaion 1-3505, Diaion 1-3521, Diaion 1-3525, Diaion 1-3529, Diaion 1-3533, Diaion 1-3541, Diaion 1-3561, Diaion 1-3565, Diaion 1-3570, Diaion 1-3573, Diaion 1-3577, Diaion 1-3581, Diaion 1-3585, and Diaion 1-3589, and Diaion 1-3593;

Dowex 50X1-100, Dowex 50X1-200, Dowex 50X1-400, Dowex 50X2-100, Dowex 50X2, 200, Dowex 50X2-400, Dowex 50X4-100, Dowex 50X4-200, Dowex 50X4-400, Dowex 50X4-200R, Dowex 50X8-100, Dowex 50X8-200, Dowex 50X8-400, Dowex-50W, Dowex 650C, Dowex D2533, Dowex D3303, Dowex D5052, Dowex G-26, Dowex G-55, Dowex I 0131, Dowex 18880, and Dowex 19880;

Duolite 1-0348, Duolite D 5427, Duolite D 5552, Duolite D 7416, and Duolite D 5677; and

DEAE-Sephacel® (Amersham Pharmacia Biotech Ltd); and DEAE Trisacryl m, SP-Trisacryl Plus-M, SP-Trisacryl M.

The concentration range of polyelectrolyte in the iontophoretic device can be from about less than 1 wt % to greater than 90 wt % of the net reservoir weight. In a preferred embodiment, the polyclectrolyte is present within the range of about 0.01-99 wt %, more preferably within the range of about 0.25-30 wt %, and most preferably within the range of about 1-30 wt % of the net reservoir weight.

IV. Compound of Interest

Compounds that can benefit from the methods of the invention include both drugs to be delivered and analytes to be extracted. These compounds can be in the form of ions, molecules, chemical compounds and compositions. In addition, one or more compounds can be delivered concurrently or extracted concurrently.

The compound of interest can be a charged or uncharged species. By altering the ionic environment within the transport pathways, ionic movement of all charged species toward the electrode with opposing polarity will be enhanced, not only the movement of charged species such as Na⁺ and Cl⁻. In a similar manner, the methods of the invention are not limited to the extraction or delivery of uncharged species towards the cathode. Similar principles apply for extraction or delivery in the direction of the anode. By placing a polyanion in the cathode (e.g., polystyrene sulfonate or dextran sulfonate), or a polycation in the anode (e.g., DEAE-dextran), convective solvent flow, or direct electrostatic movement towards those respective chambers will be significantly enhanced.

A. Analytes

The term “analyte” is used to refer to any substance that is in the system or body (e.g., circulating system, tissue system) of a patient and that can be transported across an electroporated tissue, for example a substance that can be extracted from within the patient's body, such that the substance is transported from beneath the localized region of the body surface to the exterior of the body. The extracted compounds, i.e., the analytes, can be molecular entities that are markers of disease states, pharmacologically active agents that have been administered to the subject and metabolites of such active agents, substances of abuse, electrolytes, minerals, hormones, amino acids and peptides, metal ions, nucleic acids, genes, enzymes, toxic agents, or any metabolites, conjugates, prodrugs, analogs or other derivatives (e.g., salt, ester, amide) of the aforementioned substances. In some instances, more than one substance is monitored at a time. Specific monitoring applications are described below. The substances can be charged (negatively or positively), uncharged or electronically neutral (e.g., zwitterionic substances with an equal number of opposing charges). In one embodiment, at least two analytes are extracted concurrently.

A number of different analytes that correlate with particular diseases or disease states can be monitored. Exemplary molecular entities that are markers of disease states include, by way of illustration and not limitation, glucose, galactose, lactic acid, pyruvic acid, and amino acids such as phenylalanine and tyrosine. For example, glucose is useful for monitoring diabetic patients, phenylalanine levels can be ascertained to monitor the treatment of phenylketonuria, a condition that is manifested by elevated blood phenylalanine levels, galactose levels can be ascertained for patients with galactosemia, and so forth.

For example, the invention finds particular utility when the analyte is a pharmacologically active agent whose level in the blood requires monitoring. Exemplary pharmacologically active agents include those agents that have been administered to the patient for therapeutic or prophylactic treatment, and metabolites thereof, and include, by way of illustration and not limitation, β-agonists; analeptic agents; analgesic agents; anesthetic agents; anti-angiogenic agents; anti-arthritic agents; anti-asthmatic agents; antibiotics such as aminoglycoside antibiotics; anticancer agents; anticholinergic agents; antiangiogenic agents; anticoagulant agents (e.g., heparin, low molecular weight heparin analogues, and warfarin sodium); anticoagulants; anticonvulsant agents; antidepressant agents; antidiabetic agents; antidiarrheal agents; anti-emetic agents; anti-epileptic agents; antihelminthic agents; antihistamines; antihyperlipidemic agents; antihypertensive agents; anti-infective agents; anti-inflammatory agents; antimetabolites; antimigraine agents; antiparkinsonism drugs; antipruritic agents; antipsychotic agents; antipyretic agents; antispasmodic agents; antitubercular agents; anti-ulcer agents; antiviral agents; anxiolytic agents; appetite suppressants; attention deficit disorder and attention deficit hyperactivity disorder drugs; cardiovascular agents including calcium channel blockers, antianginal agents, central nervous system (“CNS”) agents, beta-blockers and antiarrhythmic agents, for example, cardiac glycosides; central nervous system stimulants; cytotoxic drugs; diuretics; genetic materials; hormonolytics; hypnotics; hypoglycemic agents (e.g., glucagon and other carbohydrates such as glucose); immunosuppressive agents; muscle relaxants; narcotic antagonists; neuroprotective agents; nicotine; nutritional agents; parasympatholytics; peptide drugs; psychostimulants; sedatives; steroids; smoking cessation agents; sympathomimetics; photoactive agents for photodynamic therapy; tocolytic agents; tranquilizers; vasodilators; and active metabolites thereof.

Exemplary substances of abuse, include, by way of illustration and not limitation, alcohol, cannibinoids, opioids, amphetamines, benzodiazepines, and hallucinogens.

Exemplary electrolytes, include, by way of illustration and not limitation, sodium, potassium, chloride, calcium, phosphate, and blood pH.

Exemplary minerals, include, by way of illustration and not limitation, zinc, iron, copper, magnesium, potassium, and calcium.

Exemplary hormones include, by way of illustration and not limitation, growth hormone, LHRH, luteinizing hormone, insulin, and glucagon. For example, the analyte can be luteinizing hormone, which is extracted for fertility monitoring.

Exemplary amino acids and peptides, include, by way of illustration and not limitation, phenylalanine and tyrosine.

Exemplary metal ions include, by way of illustration and not limitation, zinc, iron, copper, magnesium, calcium, and zinc.

Exemplary toxic substances include, by way of illustration and not limitation, man made or natural substances that can be used, inadvertently or intentionally, to harm organisms, including humans and animals. Examples include arsenic, cyanide, acetylcholinesterase inhibitors, muscarinic compounds, nicotinic compounds, and agents of war including nerve gas, mustard gas, and biological weapons.

Exemplary compounds or substances that can be extracted for noninvasive pharmacokinetic or toxicokinetic monitoring include, by way of illustration and not limitation, anticoagulants, anti-epileptics, cardiac glycosides, amino glycoside antibiotics, antidepressants, and corticosteroids. These analytes include, by way of illustration and not limitation, warfarin, carbamazepine, phenytoin, valproic acid, gentamicin, tobramicin, amikacin, fluoxetine, paroxetine, dexamethasone, and triamcinolone.

In one embodiment of the invention, the analyte is selected from the group consisting of monosaccharides (e.g., glucose), disaccharides, oligosaccharides, organic acids (e.g., pyruvic acid and lactic acid), alcohols, fatty acids, cholesterol and cholesterol-based compounds, amino acids, zinc, iron, copper, magnesium, potassium, as well as metabolites, conjugates, prodrugs, analogs and derivatives thereof.

Additional analytes that can be extracted from humans are discussed in “Iontophoresis Devices for Drug Delivery,” by Praveen Tyle, Pharmaceutical Research, vol. 3, no. 6, pp. 318-326.

B. Drugs

The methods disclosed herein can be used in the transdermal, transocular, or transmucosal delivery of a wide range of pharmacologically active agents. The methods can generally be utilized to deliver any chemical material or compound that induces a desired pharmacological, physiological effect, and which can be iontophoretically transported across tissue. In general, pharmacologically active agents that will be iontophoretically administered using the present method will be selected from the classes of active agents described in the preceding section. Drugs or “pharmacologically active agents” that can be used in the methods and devices of the invention include those that can be delivered iontophoretically, and more specifically delivered through a localized region of a patient's body tissue.

Such drugs include agents that are therapeutically effective, prophylactically effective, or cosmeceutically effective, and can be in any suitable form such as pharmaceutically acceptable, pharmacologically active derivatives and analogs of those active agents specifically mentioned herein, including, but not limited to, salts, esters, amides, prodrugs, active metabolites, inclusion complexes, analogs, and the like. When the term “drug” is used, it is to be understood that both the active agent per se as well as pharmaceutically acceptable, pharmacologically active salts, esters, amides, prodrugs, active metabolites, inclusion complexes, analogs, etc., are included.

In some embodiments, two or more pharmacologically active agents are administered in combination, and are typically administered simultaneously. Further, a pharmacologically active agent can be combined with various agents that enhance certain aspects of transport. For instance, a first active agent can be combined with a second active agent that improves blood circulation, to enhance the rate of delivery of the therapeutic agent throughout a patient's body. Conversely, a first active agent can be combined with a second active agent that constricts local blood flow, to limit the diffusion of the compound to the general circulation and limit the first active agent's activity to the localized region of delivery. Other methods utilize one or more excipients that act to control the level of transport that occurs during the procedure.

The active agent will generally be delivered as a component of a pharmaceutical formulation suitable for topical, transdermal, transocular and/or transmucosal administration, and will contain at least one pharmaceutically acceptable vehicle. Examples of vehicles typically used in such formulations are distilled water, buffered water, physiological saline, PBS, Ringer's solution, dextrose solution, and Hank's solution. In addition, the formulation can include other carriers, adjuvants, and/or non-toxic, non-therapeutic, nonimmunogenic stabilizers, excipients and the like. The formulation may also include additional substances to approximate physiological conditions, such as pH adjusting and buffering agents, tonicity adjusting agents, wetting agents, detergents and the like. Further guidance regarding formulations that are suitable for various types of administration can be found in Remington: The Science and Practice of Pharmacy 20^{th} edition (2000).

The pharmacologically active agent delivered using the present methods is administered in an amount effective for prophylactic and/or therapeutic purposes. An effective therapeutic amount is an amount sufficient to remedy a disease state or symptoms, or otherwise prevent, hinder, retard, or reverse the progression of a disease or any other undesirable symptoms. An effective prophylactic amount is an amount sufficient to prevent, hinder or retard a disease or any other undesirable symptom. The effective amount of any particular active agent will depend upon a number of factors known to those of skill in the art, including, for example, the potency and potential toxicity of the agent, the stability of the agent in the body, and the age and weight of the patient.

The active agents can also be compounds that are not delivered for a therapeutic or prophylactic purpose, but that are otherwise physiologically or medically useful. Such compounds include, by way of example, nutrients and imaging agents.

V. Iontophoretic Devices

Because the permeability changes much slower with the present invention than with other devices, it is expected that the methods of the invention may allow for devices to accurately transport substances for at least 24 hours and up to 7 days or longer, without the need for re-calibration. Present iontophoretic devices can accurately transport substance for, in some cases, only 12 hours before needing to be replaced. Once replaced, the device will have a warm-up period to reach steady state delivery. The device may or may not need a calibration at the end of the warm-up period.

There are numerous iontophoretic devices or systems that are useful in the methods of the invention to extract an analyte from, or deliver an agent to, a patient's body beneath a localized region of body tissue. An exemplary iontophoretic delivery device typically comprises: a first electrode assembly adapted to be placed in ion-conducting and compound-delivering relation with the localized region of the body surface and comprising a reservoir housing a pharmacologically active agent to be transported into and through the body surface; a second electrode assembly adapted to be placed in ion-transmitting relation with the body surface at a location spaced apart from the first electrode assembly; and a current source electrically connected to the first and second electrode assemblies, wherein at least one of the first and second electrode assemblies further comprises a polyelectrolyte for application to the localized region of the body tissue.

Similarly, an exemplary iontophoretic extraction system typically comprises: a first electrode assembly adapted to receive an analyte and be placed in ion-conducting and analyte-receiving relation with respect to the localized region of body tissue, the assembly comprising a reservoir for collecting and containing an analyte extracted from the patient's body beneath the localized region; a second electrode assembly adapted to be placed in ion-transmitting relation with the body tissue at a location spaced apart from the first electrode assembly; and a current source electrically connected to the first and second electrode assemblies, wherein at least one of the first and second electrode assemblies further comprises a polyelectrolyte for application to the localized region of the body tissue.

Methods of using such systems would typically involve placing the first electrode assembly in contact with a body tissue and placing the second electrode assembly in contact with the body tissue at a location spaced apart from the first electrode assembly. Finally, a current is applied across the region of body tissue via the first and second electrode assemblies. The applied current is of a magnitude, voltage and duration effective to induce iontophoretic extraction of an analyte from, or delivery of an agent across the body tissue.

The following are detailed examples of iontophoretic systems useful for delivering or extracting compounds of interest using the methods described herein.

A. Delivery Systems

One embodiment of an apparatus for delivering agents across a tissue or body surface according to the methods disclosed herein, is a system comprising a first set of two electrodes, electrically connected to a power source. The power source can be a single source capable of delivering an AC or a DC signal, or include two separate sources, one for delivering an AC signal and the other that delivers a DC signal. The power source can either deliver an AC or DC signal separately or concurrently depending on the application. A circuit including the two electrodes and power source is also connected to a controller that monitors the electrical signals delivered to the electrodes and which can send signals to the power source to alter the signals transmitted therefrom.

At least one of the electrodes includes at least one reservoir and is electrically connected to a reservoir surface. Another surface of the reservoir is placed against a surface of the tissue (e.g., a patient's skin or sclera) and held in place, for example, by an adhesive or gel. If the polyelectrolyte is to be applied concurrently with the primary signal, it may be housed within the reservoir or placed on the reservoir surface. The reservoir contains one or more agents (e.g., pharmaceutical agents) that are to be delivered across the tissue. The reservoir can be a chamber that houses a solution into which the agent(s) are dissolved. Alternately, the reservoir can include a porous material that retains a solution, paste or gel containing the agent(s) to be delivered. Various other reservoir systems known to those of skill in the art can also be utilized. The other electrode of the pair is also placed in contact with a tissue surface and held in position with an adhesive or gel. This electrode is positioned to allow for formation of a current that flows between the two electrodes. When used, the offset current can be applied to drive transport of a charged agent within the reservoir across the tissue toward the electrode of opposite charge. Uncharged agents are typically driven from the anode (the positive electrode) across the tissue at physiological pH by electroosmosis.

The system may also include an optional second set or monitoring set of electrodes that are placed within the localized region of the tissue to monitor the electrical state of the tissue during transport of the agent across the tissue. As indicated above, the electrical state monitored is one that reflects the extent of tissue permeability or the state of electroporation (e.g., electrical resistance or electrical conductance). The monitoring electrodes can be separate from the first set of electrodes, although this is not required, since the first set of electrodes can also be used to monitor the electrical state of the tissue. The monitoring electrodes can be attached to a separate monitor or, optionally, to the same controller as the first set of electrodes. If attached to a separate monitor, this separate monitor can send signals regarding the electrical state of the tissue as measured by the second set of electrodes to the controller.

The first set of electrodes utilized in applying the electrical signals can be of any of the standard types of electrodes utilized in iontophoresis. Some systems use non-polarizable electrodes such as standard electrocardiograph electrodes manufactured from silver/silver chloride. Other suitable materials include gold, stainless steel and platinum. Multichannel dispersive electrodes can also be utilized in certain methods (see, e.g., Henley, U.S. Pat. No. 5,415,629).

When a DC signal or an AC signal with a DC offset signal is utilized, the electrode including the reservoir functions as either the cathode or anode depending upon the charge of the agent being delivered. In general, the anode receives the positive contribution of the offset signal, whereas the cathode receives the negative contribution of the offset signal. Consequently, with DC or AC with DC offset signals, positively charged ions are driven into the tissue at the anode and negatively charged ions are driven across the tissue at the cathode. At physiological pH, neutral agents are driven by electroosmosis into the tissue from the anode. When an offset is not utilized and only a pure AC signal is delivered, there is no formal anode or cathode.

In some systems, it can be useful to include a reservoir at both electrodes. For example, if only a pure AC signal is applied, the agent can be transported via diffusion from either reservoir. Some methods involve reversing the direction of current flow at different time points. Reservoirs located at both electrodes can be useful in such methods because delivery can occur from both reservoirs depending upon the direction of the DC or offset signal. Two reservoirs can also be utilized to good effect if two different agents of opposite charge are to be delivered. In such instances, differently charged agents are placed in separate reservoirs so that delivery can proceed simultaneously from both reservoirs. Both reservoirs may also be used to contain the polyelectrolyte.

In operation, the reservoir is filled with a solution or matrix that includes the agent to be transferred. If the reservoir includes an absorbent material, this is soaked with a solution containing the agent or coated with a paste or gel containing the agent. Once the first set of electrodes has been properly positioned, an electrical signal is delivered to the first set of electrodes via the power supply. The particular signals delivered depend upon which protocol is utilized. In general, however, the method involves utilizing the power supply to generate the primary signal of appropriate shape, duration, frequency and voltage to affect transport of the therapeutic.

For methods utilizing a prepulse, the prepulse of appropriate frequency, voltage and duration can be generated by the power source and is effective to reach an appropriate electrical state. The monitoring electrodes can be utilized during this process to follow the progress towards the desired electrical state. Once this state is achieved, a signal is sent to the controller that terminates generation of the prepulse and then generates the primary signal and/or the offset signal for application to the tissue.

As indicated above, in some methods the concentration of the agent within the reservoir is sufficiently higher than that on the other side of the tissue such that agent is transported through the localized region via passive diffusion. More typically, however, the power supply is also utilized to generate a pure DC or a DC offset signal superimposed on the AC signal. This current drives the transport of a charged agent towards the electrode having an opposite charge or a neutral agent from the anode to cathode via electroosmosis. In some procedures, the direction of the offset current flow can be reversed between the first set of electrodes to maximize the use of both electrodes and avoid the accumulation of unwanted ions/products on the surface of the electrodes or in the reservoirs.

Through the use of solid-state circuitry, the various foregoing elements such as signal delivering electrodes, power supply and reservoir can be included in a small, integrated device that can be conveniently worn by an individual without interfering with the individual's daily activities.

B. Extraction Systems

One embodiment of an apparatus for extracting substance across a tissue or body surface according to the methods disclosed herein, is a system comprising a first set of two electrodes electrically connected to a power source. The power source can be a single source capable of delivering an AC or a DC signal, or include two separate sources, one for delivering an AC signal and the other that delivers a DC signal. The power source can either deliver and AC or DC signal separately or concurrently depending on the application. A circuit including the two electrodes and power source is also connected to a controller that monitors the electrical signals delivered to the electrodes and can send signals to the power source to alter the signals transmitted therefrom.

At least one of the electrodes includes at least one reservoir and is electrically connected to a reservoir surface. Another surface of the reservoir is placed against a surface of the tissue (e.g., a patient's skin or sclera) and held in place, for example, by an adhesive or gel. The reservoir is designed to receive one or more substances (e.g., glucose, metabolites or pharmaceutical agents) that are extracted across the tissue. If the polyelectrolyte is to be applied concurrently with the primary signal, it may be housed within the reservoir or placed on the reservoir surface. The other electrode of the pair is also placed in contact with a surface of the tissue and held in position with an adhesive or gel. This electrode is positioned to allow for formation of a current that flows between the two electrodes. When only an AC signal is applied to the tissue, the direction of current flow changes direction between the two electrodes on a period equal to the frequency of the applied current. When a DC or an AC with DC offset signal is applied, current flow is in the direction to enhance the transport of a charged or uncharged substance within the system of the individual receiving treatment towards at least one reservoir across the tissue.

The apparatus can optionally include a second set or monitoring set of electrodes that are placed within the localized region of the tissue to monitor the electrical state of the tissue during extraction of the substance across the tissue. The electrical state monitored is one that reflects the extent of tissue permeability or the state of electroporation (e.g., electrical resistance or electrical conductance). This second set of electrodes is optional because the first set of electrodes can be used to monitor the electrical state of the tissue. The monitoring electrodes can be attached to a separate monitor, or optionally to the same controller as the first set of electrodes. If attached to a separate monitor, this separate monitor can send signals regarding the electrical state of the tissue as measured by the second set of electrodes to the controller.

The first set of electrodes utilized in applying, the electrical signals can be of any of the standard types of electrodes utilized in iontophoresis, as noted above.

When a DC or AC with DC offset signal is utilized, the electrode including the reservoir functions as either the cathode or anode depending upon the charge of the substance being extracted. In general, the anode receives the positive contribution of the signal, whereas the cathode receives the negative contribution of the signal. Consequently, if a DC signal or an AC with DC offset signal is applied, negatively charged ions are extracted through the tissue and received in the reservoir which is part of the anode; positively charged ions are extracted across the tissue and received in the reservoir which is part of the cathode. Because the direction of electroosmotic flow is from the anode to the cathode at physiological pH, under physiological conditions, uncharged substances are extracted across the tissue and received in the reservoir, which is part of the cathode. It should be noted that when an offset signal is not utilized and the signal only consists of an AC signal, there is no formal anode or cathode.

In some systems, it can be useful to include a reservoir at both electrodes. For example, if only an AC signal is applied, the agent can be extracted via diffusion into either reservoir. Some methods using an offset signal involve reversing the direction of current flow at different time points. Reservoirs located at both electrodes can be useful in such methods because extraction from the system of the individual into both reservoirs can occur depending upon the direction of the offset signal. Two reservoirs can also be utilized to good effect if two different substances of opposite charge, or if a neutral and a negatively charged substance, are to be extracted. In such instances, differently charged substances are extracted into separate reservoirs. Both reservoirs may also be used to contain the polyelectrolyte.

In operation, the polyelectrolyte may initially be applied to the region of tissue and then the first set of electrodes is positioned and then an electrical signal delivered to the first set of electrodes via the power supply. The particular signals delivered depend upon which protocol is utilized. The method generally involves utilizing the power supply to generate the primary signal of appropriate shape, duration, frequency and voltage to maintain a selected electrical state. If during the transport process, the electrical state deviates from the target electrical state as detected by the monitoring electrodes, then the appropriate adjustments are made with the power supply to vary the signal such that the electrical state is brought back to the target value or within the target range.

The controller can be under the control of a microprocessor. If the microprocessor determines on the basis of signals from the monitoring electrodes that the electrical state has deviated from acceptable levels, it can signal the power source to alter the signal so as to return the electrical state to the desired target. Such a controller can also include a safety shut off if it is determined that the electrical state of the tissue has reached an unacceptable level.

For methods utilizing a prepulse, the prepulse of appropriate frequency, voltage and duration is generated by the power source and is effective to reach the target electrical state. The monitoring electrodes can be utilized during this process to follow the progress towards a desired electrical state. Once this state is achieved, a signal is sent to the controller, which terminates generation of the prepulse and then generates the primary signal and/or the offset signal for application to the tissue.

In some methods the concentration of the substance within the individual's system is sufficiently higher than that on the other side of the tissue such that agent is transported through the electroporated region via passive diffusion. More typically, however, the power supply is also utilized to generate a DC or an AC with DC offset signal. This current drives the transport of a charged substance towards the electrode having an opposite charge or an uncharged substance from anode to cathode. However, in some procedures, the direction of the offset current flow is reversed between the first set of electrodes in order to reduce potential tissue irritation, prevent electrochemical depletion of the non-polarizable electrode, increase the surface area for extraction, allow the biosensor to operate for longer periods of time, and so forth.

Through the use of solid-state circuitry, the various foregoing elements such as signal extracting electrodes, power supply and reservoir can be included in a small, integrated device that can be conveniently worn by an individual without interfering with the individual's daily activities.

VI. Constant Conductance Exemplary Applications

The foregoing protocols are intended to be illustrative and not limiting in any manner. Furthermore, various aspects of these protocols can be modified and combined so as to provide a variety of different protocols for iontophoretically transporting (e.g., administering or extracting) a compound of interest across a localized region of body tissue. While the methods can be conducted with a number of different tissue types often such methods are performed with human tissue.

A. Primary Signal Only Protocol-Delivery

This exemplary method begins with the selection of a target electrical value or range (e.g., resistance or conductance). The particular target selected can vary somewhat depending upon the individual being treated and the nature of the compound being delivered. A polyelectrolyte is applied either concurrent with or followed by the primary signal (e.g., an AC signal) to reach the desired target electrical state and to facilitate delivery of the compound across the tissue. Application of the primary signal alone without a prepulse may require a longer period of time to reach the desired target. Nonetheless, application of the primary signal significantly increases transport over simple passive diffusion. Moreover, when the primary signal is an AC signal, by continually reversing polarity, the AC signal keeps the tissue depolarized and less susceptible to buildup of charged species at the surface of the tissue. The primary signal also maintains a relatively constant level of skin permeability that allows for relatively constant, controlled and predictable delivery of the agent through the tissue.

During the time of primary signal application, the electrical state of the tissue is measured, either continuously or periodically, to determine whether the electrical state of the tissue remains within the target range. If the electrical state is within the target range, the primary signal is applied without modification. If, however, the measured electrical state drifts outside the target range, then the primary signal is adjusted to return the electrical state back within specifications. The primary signal is applied for a period sufficient to deliver the desired amount of compound across the tissue at a substantially constant rate. Once the delivery period is complete, the treatment ends.

B. Primary Signal Only Protocol-Extraction

This exemplary protocol begins with the selection of a target electrical value or range. As indicated above, the particular target selected can vary depending upon the individual being treated and the nature of the substance being extracted. A polyelectrolyte is applied either concurrent with or followed by a primary signal to reach the desired target electrical state and to facilitate extraction of the substance across the tissue. Application of a primary signal alone, without a prepulse, may require a longer period to reach the desired target. Nonetheless, application of the primary signal significantly increases transport over simple passive diffusion, as pointed out earlier herein. Moreover, when the primary signal is an AC signal, by virtue of the continual reversal of polarity, the AC signal keeps the tissue depolarized and less susceptible to buildup of charged species at the surface of the tissue. The AC signal also maintains a relatively constant level of skin permeability that allows for relatively constant, controlled, predictable, and determinable extraction of a compound through the localized region of body tissue.

During the time period in which the primary signal is applied, the electrical state of the tissue is measured, either continuously or periodically, to determine whether the electrical state of the tissue remains within the target range. If the electrical state is within the target range, the signal is applied without modification. If, however, the measured electrical state drifts outside the target range, the signal is adjusted to return the electrical state back within the target range. The signal is applied for a period sufficient to extract the desired amount of substance across the tissue at a substantially constant rate after which the method ends.

C. Primary Signal Plus Prepulse Protocol-Delivery

With this exemplary protocol, the selection of a target electrical state is as described for the “Primary Signal Only Protocol-Delivery.” However, prior to or concurrent with the application of the primary signal, a polyelectrolyte and an AC and/or a DC prepulse is applied to the tissue to relatively quickly achieve the selected electrical state. Once it has been determined that the target state has been reached, the primary signal is applied to the tissue. The electrical state is monitored continuously or periodically as described in the preceding section to maintain the target electrical state throughout the time period during which delivery occurs. The primary signal is adjusted as needed to maintain the target state. Once the delivery period is completed, the procedure ends.

D. Primary Signal Plus Prepulse Protocol-Extraction

With this exemplary protocol, the selection of a target electrical state is as described for the “Primary Signal Only Protocol-Extraction.” However, prior to or concurrent with the application of the primary signal, a polyelectrolyte and an AC and/or a DC prepulse is applied to the tissue to relatively quickly achieve the selected electrical state. Once it has been determined that the target state has been reached, the primary signal is applied to the tissue. The electrical state is monitored continuously or periodically as described in the preceding section to maintain the target electrical state throughout the time period during which extraction occurs. The primary signal is adjusted as needed to maintain the target state. Once the extraction period is completed, the procedure ends.

E. Primary Signal Plus Offset Signal Protocol-Delivery

This exemplary protocol utilizes a primary signal (e.g., AC) plus the offset signal (e.g., DC) protocol. The initial stages of the method generally track those described for the “Primary Signal Only Protocol-Delivery” including selection of a target electrical state. In this particular method, the primary signal and the offset signal are applied to the tissue. The offset signal can be applied simultaneously with the application of the primary signal or at any time during the treatment. If it is determined that the electrical state is no longer at the targeted value, the primary signal is adjusted to return the electrical state to the target value or range. Such an adjustment is usually independent to the offset signal and does not affect the offset signal driven transport. The offset signal is typically kept constant but can optionally be adjusted during the application period to change the delivery rate of the agent being transferred. Once a desired amount of agent has been delivered or the time period of treatment has expired, application of the primary and offset signals is terminated.

F. Primary Signal Plus Offset Signal Protocol-Extraction

This exemplary protocol utilizes a primary signal (e.g., AC) plus the offset signal (e.g., DC) protocol. The initial stages of the method generally track those described for the “Primary Signal Only Protocol-Extraction” including selection of a target electrical state. In this particular method, however, a polyelectrolyte, the primary signal, and the offset signal are applied to the tissue. The offset signal can be applied simultaneously with the application of the primary signal or any time during the treatment period. If it is determined that the electrical state is no longer at the targeted value, the primary signal is adjusted to return the electrical state to the target value or range. Such an adjustment is usually independent of the offset signal and is generally non-interfering with the offset signal driven transport. The offset signal is typically kept constant but can optionally be adjusted to change the extraction rate of the substance being transferred during the treatment. Once the desired amount of substance has been extracted, application of the primary and offset signals is terminated.

G. Primary Signal Plus Prepulse Plus Offset Signal Protocol-Delivery

This exemplary protocol combines the prepulse and the offset signals with the primary signal. Such methods utilize the unique features of each type of signal to optimize delivery of an agent. As described above, a target electrical state is selected followed by application of a polyelectrolyte and the prepulse signal to quickly establish a selected electrical state correlated with an increased level of tissue permeability that promotes delivery of the agent. Once it is determined that the target state has been reached, the primary signal and the offset signal are applied, with the primary signal mainly functioning to maintain the target electrical state and the offset signal acting to promote delivery of the compound across the electroporated tissue. The electrical state is monitored, and if the electrical state is found to vary from the target, the primary signal is adjusted as required to return the electrical state to the target. Once the treatment time has elapsed, the process is completed.

H. Primary Signal Plus Prepulse Plus Offset Signal Protocol-Extraction

This exemplary protocol combines the prepulse and the offset signals with the primary signal. Such methods utilize the unique features of each type of signal to optimize extraction of a substance. As described above, a target electrical state is selected followed by application of a polyelectrolyte and the prepulse signal to quickly establish a selected electrical state correlated with an increased level of tissue permeability that promotes extraction of the substance. Once it is determined that the target state has been reached, the primary signal and offset signal are applied, with the primary signal mainly functioning to maintain the target electrical state and the offset signal acting to promote extraction of the compound across the electroporated tissue. The electrical state is monitored, and if the electrical state is found to vary from the target, the primary signal is adjusted as required to return the electrical state to the target. Once the desired amount of substance has been extracted, the process is completed.

VII. Exemplary Analytical Processes Following Extraction

The analyte extraction methods provided herein can also be used in a variety of applications, including the diagnosis and monitoring of various disorders and diseases, e.g., in monitoring a patient's glucose level on a periodic or substantially continuous basis. Instead of monitoring glucose levels directly, one can monitor a product formed during metabolism of glucose such as lactic acid and/or pyruvic acid. In addition, the method of the invention can be used to detect or monitor the presence of a substance within an individual's system that is correlated with a particular disease or disease state (i.e., a disease “marker”). For example, phenylalanine can be extracted from the body of a patient with phenylketonuria in order to detect whether phenylalanine is accumulating in the patient's blood, or it can be extracted as part of a screening method to monitor phenylalanine levels so as to assess risks for or the presence of phenylketonuria. Another example is the monitoring of blood alcohol or illicit substances as part of a court ordered treatment program. Yet another example is the monitoring of toxic substance levels in the body as a means for monitoring an individual's exposure to hazardous materials.

The extraction methods also have utility in a variety of therapeutic applications. By way of example, the level of one or more pharmacologically active agents or metabolites thereof in a patient's body can be tracked as a way to assess the current levels of active agent within the patient's system and adjust dosage or dosing regimen, as necessary. This is particularly relevant when a patient is receiving drugs that have a narrow therapeutic window, such as aminoglycoside antibiotics, antiepileptic agents, cardiac glycosides, and anticoagulants. This is also relevant when the patient is receiving cytotoxic or immunosuppressant drugs.

In yet a further embodiment, the tracking of a patient's blood level of a therapeutic agent can be coupled with a drug delivery device to automatically maintain the blood level of the active agent within a narrow therapeutic window. Thus, in such embodiments, certain systems of the invention, can include a reservoir at one electrode for collecting the analyte extracted from the patient's body and a second reservoir at the second electrode for delivering the active agent. As a specific example, one iontophoretic system of the invention can be used both to extract glucose to monitor a patient's glucose level and to deliver insulin or another hypoglycemic agent as needed.

The presence of a particular compound of interest in the reservoir can be detected utilizing a variety of techniques. For example, if a liquid is collected within the reservoir, the presence of one or more compounds of interest within the liquid can be detected using any of a variety of analytical techniques such as various chromatographic methods (e.g., high performance liquid chromatography), spectroscopic methods (e.g., infra-red spectroscopy, nuclear magnetic resonance spectroscopy and mass spectroscopy), electrochemical methods (e.g., electrical resistance and/or electrical potential), and enzymatic methods coupled with colorimetric analysis or electrical potential changes. Combinations of analytical techniques can also be utilized (e.g., gas chromatography/mass spectroscopy). Detection of the substance can be either qualitative or quantitative.

The reservoir can include various agents that specifically react with one or more compounds of interest to form a detectable product or complex. For example, the reservoir can include a dye that emits or absorbs light of a particular wavelength upon interaction with a particular compound. Alternatively, an enzyme with specific activity for the analyte can be coupled to another enzyme with specific activity for another ligand capable of releasing electrons detectable by a sensor when metabolized by the second enzyme. For example, if the extracted substance is glucose, the enzyme can be glucose oxidase. The glucose oxidase can be coupled with peroxidases, which cause electron release that can be detected by a sensor. Various other sensors can be utilized to detect glucose, such as glucose selective electrodes (see, e.g., Solsky, Anal. Chem. 60:106R-113R (1988)) and various in situ analyses known in the art (e.g., calorimetric analyses).

The concentration of a substance in the extraction reservoir can be correlated with the concentration of the substance amount or concentration of the substance in the patient's body in various ways. In some instances, mathematical algorithms established from a large population set or calibration procedures are utilized to correlate the two values.

VIII. Exemplary Therapeutic Applications

The methods and iontophoretic devices provided herein can be used in a variety of applications for the delivery of compounds, including the treatment of various disorders and diseases. Certain methods are used in the treatment of diabetes and various weight disorders such as obesity, for example. In the treatment of diabetes, the methods can be used for the controlled delivery of insulin or other hypoglycemic agents, and in the administration of glucagon or other carbohydrates (e.g., glucose) to an individual who is hypoglycemic. Weight loss treatments can involve the delivery of appetite suppressors such as cholecystokinin, for example.

Related transport methods are performed to assist in treating individuals seeking to recover from narcotic or other types of substance abuse. These methods can involve, for example, the administration of agents that assist in the detoxification process. The delivery methods also find value in treating nicotine addiction. Treatment of nicotine addiction often involves a program in which decreasing levels of nicotine are delivered over an extended treatment period. Detoxification methods generally involve iontophoretic delivery of an agent that blocks the effect of, or substitutes for, the substance being abused.

Certain transport methods lend themselves well to the treatment of various blood circulation and pressure disorders. For example, the methods can be used in the iontophoretic delivery of various anticoagulants (e.g., heparin, low molecular weight heparin analogues, and warfarin sodium). Such methods can be useful in prevention of stroke and/or in the reducing clotting risk following certain surgical procedures. Treatment of blood pressure disorders is effected by the delivery of appropriate levels of blood pressure medicines, such as α-receptor blocking agents (“α-blockers”) and β-receptor blocking agents (“β-blockers”). The method of the invention is also useful in pain management, i.e., in the iontophoretic delivery of various analgesic agents to control pain during surgery or in ongoing pain management. The method of the invention is also useful in the treatment of migraine headaches and in acute or chronic nausea. The method of the invention may also be used in the iontophoretic administration of drugs for treating psychiatric disorders, sleep disorders, movement disorders (e.g. Parkinson's disease), infections, and local and diffuse inflammatory disorders.

The present method is also useful in treating local rather than systemic conditions and disorders. For example, the method may be used to effect iontophoretic delivery of active agents appropriate for treating skin conditions such as acne, eczema and psoriasis, local inflammation, microbial infections and the like. The invention may also be implemented in the fields of cosmetics and cosmeceuticals, for example, in hydrating the skin or in removing the external layer of the skin, thereby stimulating the activation of various collagen growth factors and the growth of new skin layers. This method may further be useful in the treatment of skin malignancies or warts by pinpoint delivery of photodynamic therapy agent (PDT) to a skin lesion with subsequent activation by the appropriate wavelength of light.

### EXAMPLES

The practice of the present invention will employ, unless otherwise indicated, conventional techniques of pharmaceutical formulation, and the like, which are within the skill of the art. Such techniques are explained fully in the literature. Preparation of various types of pharmaceutical formulations are described, for example, in Remington: The Science and Practice of Pharmacy, Nineteenth Edition. (1995) and Ansel et al., Pharmaceutical Dosage Forms and Drug Delivery Systems, 6^{th} Ed. (Media, Pa.: Williams & Wilkins, 1995).

The following examples are put forth so as to provide those of ordinary skill in the art with a complete disclosure and description of how to make and use the compounds of the invention, and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. and pressure is at or near atmospheric. All components were obtained commercially unless otherwise indicated.

Materials

Conductive silver paint was purchased from Ladd Research Technologies (Williston, Vt.) and silver foil from EM-Science (Gibbstown, N.J.). Silver chloride powder, phosphate buffered saline (PBS, pH 7.4) tablets and agarose were purchased from Sigma (St. Louis, Mo.). Dextran sulfate, sodium salt (average molecular weight 500,000, DS500K) was purchased from Polysciences, Inc., (Warrington, Pa.). Polystyrene sulfonate standards (1,300 and 18,000 with a narrow polydispersity with a M_{w}/Mₙ of 1.2) were purchased from Polysciences, Inc., (Warrington, Pa.). ¹⁴C-Mannitol was purchased from Moravek (Brea, Calif.) or American Radiochemical Corp (St. Louis, Mo.). Ultimate Gold® scintillation cocktail was purchased from Packard (Meriden, Conn.) and liquid scintillation counting was performed by a Packard TriCarb Model 1900 TR liquid scintillation analyzer. A custom built AC waveform generator power supply (EM-Tech electronics, Lindon Utah) or Phoresor-II PM 700 (Iomed, Inc., Salt Lake City, Utah) was used as the iontophoretic power supply. Human epidermal membrane was obtained from licensed sources and experiments were conducted under local RB approval. All water was >18 MΩ prepared by the Milli-Q process.

Methods

All of the following experiments were conducted using a side-by-side type diffusion cell with an open diffusional area of 0.85 cm². The cells were separated by a piece of dermatomed, heat-separated human epidermal membrane with the stratum corneum facing the receiver compartment. Each side of the diffusion cell had a 2 ml volume and was stirred at 350 rpm with a magnetic stir bar.

The electrodes were prepared by dipping a silver foil strip into a 1:1 (w/w) mixture of conductive silver paint and finely ground silver chloride. After dipping, the electrodes were hung and allowed to cure at room temperature overnight.

The receiver compartment was filled with either PBS or the 1.67% (w/v) polyelectrolyte DS500K agent. In Example 1 the donor compartment contained PBS spiked with 1 μCi/ml ¹⁴C-mannitol. In Example 2, the donor compartment contained PBS spiked with 30 μl ¹⁴C-mannitol/ml.

### Example 1

A 1000 Hz AC potential with a 0.1 mA DC offset was applied to decrease the skin 2 resistance to 4 kΩ cm² and continued for 8 hours. Every 20 minutes, the entire volume of the receiver solution was removed, mixed with scintillation cocktail, and analyzed by liquid scintillation counting. All experiments were conducted in nonuplicate.

The amount of ¹⁴C-mannitol transported across the membrane was plotted as a function of time. Permeabilities were determined from the following equations:

J=ΔQ/AΔt

where J is the flux, Q is the amount of solute transported across the membrane, A is the area of the exposed membrane and t is the time.

P=J/C_{D}

where P is the permeability and C_{D} is the concentration of the solute in the donor solution.

Permeabilities were plotted as a function of time and the slope of the best-fit line to the steady state portion of the curve was determined using regression analysis. All statistical analysis was accomplished using the statistical analysis package bundled with Microsft™ Excel. Experimental results for the nine runs are presented in Table 1. The lag-time was calculated by extrapolating a best-fit line of the steady state portion of the cumulative d.p.m. versus time curve, back to the x axis (zero d.p.m.). The terminal slope of the permeability versus time curve, was calculated by linear regression of the last 10 data points. Statistical significance was determined by analysis of variance (ANOVA).

TABLE 1

| | | | Terminal Slope | |
|---|---|---|---|---|
| | Lag Time (min) | | of P vs. Time Curve | |
| Run # | PBS/PBS | PBS/DS500K | PBS/PBS | PBS/DS500K |
| 1 | 119.8 | 74.6 | 4.30E−11 | 7.03E−11 |
| 2 | 123.9 | 57.5 | 4.46E−11 | −1.89E−11 |
| 3 | 156.9 | 121.0 | 3.89E−11 | 5.05E−11 |
| 4 | 176.9 | 107.0 | 5.33E−11 | 9.08E−12 |
| 5 | 185.6 | 116.9 | 5.53E−11 | 3.68E−11 |
| 6 | 166.0 | 33.9 | 5.52E−11 | 1.88E−11 |
| 7 | 131.9 | 55.9 | 6.06E−11 | −7.48E−11 |
| 8 | 143.6 | 136.3 | 4.60E−11 | 2.94E−11 |
| 9 | 59.6 | 85.6 | 4.10E−11 | 1.55E−11 |
| Average | 140.8 | 87.7 | 4.86E−11 | 1.52E−11 |

| | | |
|---|---|---|
| Statistical p | 0.007 | 0.03 |

The permeability of mannitol through the human epidermal membrane was two- to three-fold higher with the DS500K-containing system as compared to the PBS only system (Control). In addition, the DS500K-containing system reached the plateau quicker as compared to the Control, typically within two hours, while the permeability in the Control continued to steadily increase throughout the eight-hour run. Analysis of the cumulative d.p.m. versus time plots revealed an average lag-time of 140 minutes for the Control, with only an 87 minute average lag-time for the DS500K-containing system, a statistically significant difference.

Table 1 also presents the slope of a best-fit line through the terminal point (last 10 data points) of the permeability versus time curve. The smaller the change in this slope, the smaller the intrasubject variability and the smaller the “flux-drift” observed by other DC iontophoretic devices. In the Control, the flux drift was over three-fold higher than the flux drift for the DS500K-containing system (P=0.03). Thus, the inclusion of a polyelectrolyte in an iontophoretic device with exclusion of PBS decreases lag-time and increases transport reproducibility as treatments within an individual progress.

### Example 2

The negatively charged cathode was placed into a reservoir containing either PBS or the 1.67% (w/v) polyclectrolyte DS500K agent. The reservoir was connected to the receiver chamber with a salt bridge containing 2% agarose and the polyelectrolyte or PBS. The salt bridge was necessary to impede the transport of Cl⁻ into the receiver chamber that was electrochemically liberated from the cathode by the passage of the electrical current. The positively charged anode was placed in the donor compartment. A human epidermal membrane separated the donor compartment and the receiver chamber. A current of 0.1 mA was passed between the two electrodes during the experiment.

Each experiment was run for 3 consecutive days. On day 1, the experiment was conducted with PBS in the donor chamber, the salt bridge, the reservoir, and the receiver chamber. On day 2, the PBS in the reservoir, salt bridge, and receiver chamber was replaced with the polyelectrolyte. This allowed each piece of membrane to serve as its own control. Day 3 again saw PBS in both electrode chambers and served as a control to ensure that the polyelectrolyte did not evince its enhancement through irreversible perturbation of the membrane. In all cases, the permeability from day 3 was not statistically different than day 1. The day 3 results have, therefore, been omitted for clarity.

Every 45 minutes during the experimental run, 100 μl of the receiver solution was withdrawn and mixed with 10 ml of scintillation cocktail. Permeability was calculated from the cumulative dpm vs. time plot. All experiments were run in at least triplicate. The results from the above-described experimental examples are presented in Tables 2 and 3.

Table 2 shows the measurement of mannitol electroosmotic flux enhancement between PBS as the extraction medium and the polyelectrolyte agent as the extraction medium during the first 2¼ hours. The normalized cumulative amount is the cumulative DPM at 135 minutes in the receiver chamber divided by the DPM initially present in the donor chamber. “PSS” refers to polystyrene sulfonate.

TABLE 2

| | | Mean | Mean Normalized | |
|---|---|---|---|---|
| | | Normalized | Cumulative | |
| | Polyelectrolyte | Cumulative | Amount with | Enhance- |
| | Concentration | Amount in | Polyelectrolyte | ment |
| Exp # | (% w/v) | PBS | (cm/s) | Factor |
| 1 | PSS 1,300/13% | 0.012 | 0.113 | 9.4 |
| 2 | PSS 18,000/13% | 0.036 | 1.033 | 28.7 |
| 3 | PSS 18,000/2% | 0.044 | 0.125 | 2.9 |
| 4 | Dextran Sulfate/ | 0.039 | 0.146 | 3.8 |
| | 1.67% | | | |
| 5 | Dextran Sulfate/ | 0.024 | 0.136 | 5.6 |
| | 0.8% | | | |

Table 3 provides data for the intersample variability for mannitol flux as measured by the standard error of the mean (SEM) of the steady state permeability. The standard error of the mean is the standard deviation normalized for the mean ((Standard Deviation/Mean)*100%). N=3 for each experiment.

TABLE 3

| | Polyelectrolyte | Mean Steady State | Mean Steady State |
|---|---|---|---|
| | Concentration | PBS Permeability | Permeability with |
| Exp # | (% w/v) | SEM | Polyelectrolyte SEM |
| 1 | PSS 1,300/13% | 81.5% | 37.2% |
| 2 | PSS 18,000/13% | 62.3% | 33.1% |
| 3 | PSS 18,000/2% | 66.8% | 45.1% |
| 4 | Dextran Sulfate/1.67% | 29.6% | 64.5% |
| 5 | Dextran Sulfate/0.8% | 55.9% | 16.2% |

From Table 2 above, it is evident that when chloride ions are replaced by large polyelectrolyte ions in the receiver compartment, the electroosmotic flux of mannitol towards the receiver chamber substantially increases, with the average enhancement ranging from almost 3 to 29 fold. From this example, it is clear that the present invention provides an important advantage over the art.

In addition, with the exception of 1.67% dextran sulfate, Table 3 demonstrates that replacement of chloride with a large polyelectrolyte substantially reduces the inter-sample variability as measured by the standard error of the mean. The replacement of the highly mobile chloride ion by the relatively immobile polyelectrolyte improves the variability in the permeability observed between subjects, often by two-fold or more.

All patents, publications, and other published documents mentioned or referred to in this specification are herein incorporated by reference in their entirety.

It is to be understood that while the invention has been described in conjunction with the preferred specific embodiments hereof, the foregoing description, as well as the examples which are intended to illustrate and not limit the scope of the invention, it should be understood by those skilled in the art that various changes may be made and equivalents may be substituted without departing from the scope of the invention. Other aspects, advantages and modifications will be apparent to those skilled in the art to which the invention pertains.

Accordingly, the scope of the invention should therefore be determined with reference to the appended claims, along with the full range of equivalents to which those claims are entitled.

We claim:

1. A method of decreasing flux variability in an iontophoretic device used to transport a compound of interest through a localized region of a patient's body tissue, comprising:
(a) applying a current to the localized region of body tissue at a level sufficient to effect iontophoretic transport of the compound of interest therethrough;
(b) either prior to, during, or both prior to and during application of the current, applying to the localized region of body tissue an amount of at least one polyelectrolyte effective to stabilize the rate of flux of the compound of interest through the localized region of body tissue.

2. The method of claim 1 wherein the polyelectrolyte has a molecular weight of about 200 Da or greater.

3. The method of claim 2 wherein the polyelectrolyte has a molecular weight within the range of about 200-1000 Da.

4. The method of claim 2 wherein the polyelectrolyte has a molecular weight within the range of about 1000-10,000 Da.

5. The method of claim 2 wherein the polyelectrolyte has a molecular weight greater than 10,000 Da.

6. The method of claim 1 wherein the polyelectrolyte is selected from the group consisting of cationic polyelectrolytes, anionic polyelectrolytes, nonionic polyelectrolytes, amphoteric polyelectrolytes, and mixtures thereof.

7. The method of claim 6 wherein the polyelectrolyte is selected from the group consisting of cationic polyelectrolytes, anionic polyelectrolytes, and amphoteric polyelectrolytes, and comprises at least one ionic group selected from the group consisting of sulfonates, carboxylates, phosphates, and quaternary ammonium groups.

8. The method of claim 6 wherein the polyelectrolyte is selected from the group consisting of acrylamides, addition polymers, oligosaccharides and polysaccharides, polyamines, polycarboxylic acid salts, polyethylenes, polyimines, polystyrenes, and mixtures thereof.

9. The method of claim 6 wherein the polyelectrolyte is a cationic polyelectrolyte.

10. The method of claim 9 wherein the cationic polyelectrolyte comprises an ionic group selected from the group consisting of quaternary ammonium; primary, secondary, or tertiary amines charged at the reservoir solution pH; heterocyclic compounds charged at reservoir solution pH; sulfonium; and phosphonium groups.

11. The method of claim 10 wherein the cationic polyelectrolyte is selected from the group consisting of addition polymers, aminated styrenes, cholestyramine, polyimines, aminated polysaccharides, and mixtures thereof.

12. The method of claim 6 wherein the polyelectrolyte is an anionic polyelectrolyte.

13. The method of claim 12 wherein the anionic polyelectrolyte comprises an anion selected from the group consisting of carboxylate, sulfonate and phosphate groups.

14. The method of claim 13 wherein the anionic polyelectrolyte is selected from the group consisting of acrylamides, alginate, alginic acid, addition polymers, hyaluronate, oligosaccharides, pectic acid, polyacrylic acids, polysaccharides, polystyrenesulfonic acids, polyvinylphosphonic acids, and mixtures thereof.

15. The method of claim 6 wherein the polyelectrolyte is an amphoteric polyelectrolyte.

16. The method of claim 1 wherein the polyelectrolyte is selected from the group consisting of heparin and heparin derivatives, anionic and cationic liposomes, anionic and cationic micelles, polyamines, polyethylenes, polysaccharides, and mixtures thereof.

17. The method of claim 16 wherein the polysaccharide is selected from the group consisting of agaroses, celluloses, dextrans, and starch.

18. The method of claim 1 wherein the polyelectrolyte is an ion exchange material.

19. The method of claim 18 wherein the ion exchange material is selected from the group consisting of polyacrylic acids, polyacrylic sulfonic acids, polyacrylic phosphoric acids and polyacrylic glycolic acids, polyvinyl amines, polystyrenes, poly epichlorohydrin/tetraethylenetriamines, and polymers having pendent amine groups.

20. The method of claim 18 wherein the ion exchange material is a strongly acidic cation exchange resin.

21. The method of claim 18 wherein the ion exchange material is a weakly acidic cation exchange resin.

22. The method of claim 18 wherein the ion exchange material is a strongly basic anion exchange resin.

23. The method of claim 18 wherein the ion exchange material is a weakly basic anion exchange resin.

24. The method of claim 18 wherein the ion exchange material is a mixed bed resin.

25. The method of claim 1 wherein the polyelectrolyte comprises from about less than 1 wt % to greater than 90 wt % of the net reservoir weight.

26. The method of claim 25 wherein the polyelectrolyte comprises about 0.01-99 wt % of the net reservoir weight.

27. The method of claim 26 wherein the polyelectrolyte comprises about 0.25-30 wt % of the net reservoir weight.

28. The method of claim 1 wherein the iontophoretic device further comprises a membrane positioned between the polyelectrolyte and the localized region of body tissue, wherein the membrane has a pore size sufficient to prevent transport of polyclectrolyte therethrough and sufficient to permit transport of the compound of interest therethrough.

29. The method of claim 1 wherein the current is an alternating current.

30. The method of claim 29 wherein the current is applied to the localized region of the body tissue for a time period within the range of approximately 2 minutes to greater than 72 hours.

31. The method of claim 30 wherein the time period is within the range of approximately 12-72 hours.

32. The method of claim 29 wherein the current is applied at a voltage level within the range of about 1-75 V.

33. The method of claim 32 wherein the voltage level is within the range of about 1-45 V.

34. The method of claim 29 which further comprises applying a direct current prepulse prior to step (a).

35. The method of claim 29 which further comprises superimposing a direct current over the alternating current during step (a).

36. The method of claim 1 wherein the current is a direct current.

37. The method of claim 36 wherein the current is applied to the localized region of the body tissue for a time period within the range of approximately 2 minutes to greater than 72 hours.

38. The method of claim 37 wherein the time period is within the range of approximately 12-72 hours.

39. The method of claim 36 wherein the current is applied at a level within the range of about 0.01-0.5 mA/cm².

40. The method of claim 39 wherein the current is applied at a level within the range of about 0.1-0.5 mA/cm².

41. The method of claim 1 wherein the polyelectrolyte is applied to the localized region of body tissue prior to application of the current.

42. The method of claim 1 wherein the polyelectrolyte is applied to the localized region of body tissue during application of the current.

43. The method of claim 1 wherein the polyelectrolyte is applied to the localized region of body tissue both prior to and during application of the current.

44. The method of claim 1 wherein the body tissue is skin.

45. The method of claim 1 wherein the body tissues is ocular tissue

46. The method of claim 45 wherein the ocular tissue is selected from the group consisting of conjunctiva, sclera and cornea.

47. The method of claim 1 wherein the body tissue is mucosal tissue.

48. The method of claim 1 wherein the localized region of body tissue has an area within the range of about 0.1-100 cm².

49. The method of claim 1 that provides for at least a 25% decrease in variability in the flux compared to the variability in the flux in the absence of the polyelectrolyte.

50. The method of claim 49 that provides for at least a 50% decrease in variability in the flux compared to the variability in the flux in the absence of the polyelectrolyte.

51. The method of claim 50 that provides for at least a 75% decrease in variability in the flux compared to the variability in the flux in the absence of the polyelectrolyte.

52. The method of claim 49 wherein the decreased variability is expressed as decreased intrasubject variability.

53. The method of claim 49 wherein the decreased variability is expressed as decreased intersubject variability.

54. The method of claim 1 which further provides for at least a 50% enhanced flux of the compound of interest compared to the flux in the absence of the polyelectrolyte.

55. The method of claim 54 which further provides for at least a 100% enhanced flux of the compound of interest compared to the flux in the absence of the polyelectrolyte.

56. The method of claim 55 which further provides for at least a 200% enhanced flux of the compound of interest compared to the flux in the absence of the polyelectrolyte.

57. The method of claim 1 wherein the compound of interest is a charged species.

58. The method of claim 1 wherein the compound of interest is an uncharged species.

59. The method of claim 1 wherein the compound of interest is an analyte extracted from within the patient's body, such that analyte is transported from beneath the localized region to the exterior of the body.

60. The method of claim 59 wherein the analyte is selected from the group consisting of glucose, galactose, lactic acid, pyruvic acid, and amino acids.

61. The method of claim 60 wherein the amino acid is selected from the group consisting of phenylalanine and tyrosine.

62. The method of claim 59 wherein the analyte is selected from the group consisting of diseases state markers, pharmacologically active agents, substances of abuse, electrolytes, minerals, hormones, amino acids, peptides, metal ions, nucleic acids, genes, enzymes, toxic agents, metabolites, conjugates, prodrugs, analogs and derivatives thereof.

63. The method of claim 59 wherein the analyte is selected from the group consisting of monosaccharides, disaccharides, oligosaccharides, organic acids, alcohols, fatty acids, cholesterol and cholesterol-based compounds, amino acids, zinc, iron, copper, magnesium, potassium, and metabolites, conjugates, prodrugs, analogs and derivatives thereof.

64. The method of claim 59 wherein the analyte is a pharmacologically active agent that has been administered to the patient.

65. The method of claim 64 wherein the pharmacologically active agent is selected from the group consisting of β-agonists; analeptic agents; analgesic agents; anesthetic agents; anti-angiogenic agents; anti-arthritic agents; anti-asthmatic agents; antibiotics; anticancer agents; anticholinergic agents; anticoagulant agents; anticonvulsant agents; antidepressant agents; antidiabetic agents; antidiarrheal agents; anti-emetic agents; anti-epileptic agents; antihelminthic agents; antihistamines; antihyperlipidemic agents; antihypertensive agents; anti-infective agents; anti-inflammatory agents; antimetabolites; antimigraine agents; antiparkinsonism drugs; antipruritic agents; antipsychotic agents; antipyretic agents; antispasmodic agents; antitubercular agents; anti-ulcer agents; antiviral agents; anxiolytic agents; appetite suppressants; attention deficit disorder and attention deficit hyperactivity disorder drugs; cardiovascular agents; central nervous system stimulants; cytotoxic drugs; diuretics; genetic materials; hormonolytics; hypnotics; hypoglycemic agents; immunosuppressive agents; muscle relaxants; narcotic antagonists; neuroprotective agents; nicotine; nutritional agents; parasympatholytics; peptide drugs; psychostimulants; sedatives; steroids; smoking cessation agents; sympathomimetics; photoactive agents; tocolytic agents; tranquilizers; vasodilators; and active metabolites thereof.

66. The method of claim 65 wherein at least two analytes are extracted concurrently.

67. The method of claim 1 wherein the compound of interest is a pharmacologically active agent to be delivered into the patient's body.

68. The method of claim 67 wherein the pharmacologically active agent is selected from the group consisting of β-agonists; analeptic agents; analgesic agents; anesthetic agents; anti-angiogenic agents; anti-arthritic agents; anti-asthmatic agents; antibiotics; anticancer agents; anticholinergic agents; anticoagulant agents; anticonvulsant agents; antidepressant agents; antidiabetic agents; antidiarrheal agents; anti-emetic agents; anti-epileptic agents; antihelminthic agents; antihistamines; antihyperlipidemic agents; antihypertensive agents; anti-infective agents; anti-inflammatory agents; antimetabolites; antimigraine agents; antiparkinsonism drugs; antipruritic agents; antipsychotic agents; antipyretic agents; antispasmodic agents; antitubercular agents; anti-ulcer agents; antiviral agents; anxiolytic agents; appetite suppressants; attention deficit disorder and attention deficit hyperactivity disorder drugs; cardiovascular agents; central nervous system stimulants; cytotoxic drugs; diuretics; genetic materials; hormonolytics; hypnotics; hypoglycemic agents; immunosuppressive agents; muscle relaxants; narcotic antagonists; neuroprotective agents; nicotine; nutritional agents; parasympatholytics; peptide drugs; psychostimulants; sedatives; steroids; smoking cessation agents; sympathomimetics; photoactive agents; tocolytic agents; tranquilizers; vasodilators; and active metabolites thereof.

69. The method of claim 67 wherein at least two pharmacologically active agents are administered simultaneously.

70. A method of decreasing lag time of the iontophoretic transport of a compound of interest through a localized region of a patient's body tissue, comprising:
(a) applying a current to the localized region of body tissue at a level sufficient to effect iontophoretic transport of the compound of interest therethrough;
(b) either prior to, during, or both prior to and during application of the current, applying to the localized region of body tissue an amount of at least one polyelectrolyte effective to decrease the time needed to achieve steady state transport of the compound of interest through the localized region of body tissue.

71. The method of claim 70 wherein the polyelectrolyte has a molecular weight of about 200 Da or greater.

72. The method of claim 71 wherein the polyelectrolyte has a molecular weight within the range of about 200-1000 Da.

73. The method of claim 71 wherein the polyelectrolyte has a molecular weight within the range of about 1000-10,000 Da.

74. The method of claim 71 wherein the polyelectrolyte has a molecular weight greater than 10,000 Da.

75. The method of claim 70 wherein the polyclectrolyte is selected from the group consisting of cationic polyelectrolytes, anionic polyelectrolytes, nonionic polyelectrolytes, amphoteric polyelectrolytes, and mixtures thereof.

76. The method of claim 75 wherein the polyelectrolyte is selected from the group consisting of cationic polyelectrolytes, anionic polyelectrolytes, and amphoteric polyelectrolytes, and comprises at least one ionic group selected from the group consisting of sulfonates, carboxylates, phosphates, and quaternary ammonium groups.

77. The method of claim 75 wherein the polyelectrolyte is selected from the group consisting of acrylamides, addition polymers, oligosaccharides and polysaccharides, polyamines, polycarboxylic acid salts, polyethylenes, polyimines, polystyrenes, and mixtures thereof.

78. The method of claim 75 wherein the polyelectrolyte is a cationic polyclectrolyte.

79. The method of claim 78 wherein the cationic polyelectrolyte comprises an ionic group selected from the group consisting of quaternary ammonium; primary, secondary, or tertiary amines charged at the reservoir solution pH; heterocyclic compounds charged at reservoir solution pH; sulfonium; and phosphonium groups.

80. The method of claim 79 wherein the cationic polyelectrolyte is selected from the group consisting of addition polymers, aminated styrenes, cholestyramine, polyimines, aminated polysaccharides, and mixtures thereof.

81. The method of claim 75 wherein the polyelectrolyte is an anionic polyelectrolyte.

82. The method of claim 81 wherein the anionic polyelectrolyte comprises an anion selected from the group consisting of carboxylate, sulfonate and phosphate groups.

83. The method of claim 82 wherein the anionic polyelectrolyte is selected from the group consisting of acrylamides, alginate, alginic acid, addition polymers, hyaluronate, oligosaccharides, pectic acid, polyacrylic acids, polysaccharides, polystyrenesulfonic acids, polyvinylphosphonic acids, and mixtures thereof.

84. The method of claim 75 wherein the polyelectrolyte is an amphoteric polyclectrolyte.

85. The method of claim 70 wherein the polyclectrolyte is selected from the group consisting of heparin and heparin derivatives, anionic and cationic liposomes, anionic and cationic micelles, polyamines, polyethylenes, polysaccharides, and mixtures thereof.

86. The method of claim 85 wherein the polysaccharide is selected from the group consisting of agaroses, celluloses, dextrans, and starch.

87. The method of claim 70 wherein the polyelectrolyte is an ion exchange material.

88. The method of claim 87 wherein the ion exchange material is selected from the group consisting of polyacrylic acids, polyacrylic sulfonic acids, polyacrylic phosphoric acids and polyacrylic glycolic acids, polyvinyl amines, polystyrenes, poly epichlorohydrin/tetraethylenetriamines, and polymers having pendent amine groups.

89. The method of claim 87 wherein the ion exchange material is a strongly acidic cation exchange resin.

90. The method of claim 87 wherein the ion exchange material is a weakly acidic cation exchange resin.

91. The method of claim 87 wherein the ion exchange material is a strongly basic anion exchange resin.

92. The method of claim 87 wherein the ion exchange material is a weakly basic anion exchange resin.

93. The method of claim 87 wherein the ion exchange material is a mixed bed resin.

94. The method of claim 70 wherein the polyelectrolyte comprises from about less than 1 wt % to greater than 90 wt % of the net reservoir weight.

95. The method of claim 94 wherein the polyelectrolyte comprises about 0.01-99 wt % of the net reservoir weight.

96. The method of claim 95 wherein the polyelectrolyte comprises about 0.25-30 wt % of the net reservoir weight.

97. The method of claim 70 wherein the iontophoretic device further comprises a membrane positioned between the polyelectrolyte and the localized region of body tissue, wherein the membrane has a pore size sufficient to prevent transport of polyclectrolyte therethrough and sufficient to permit transport of the compound of interest therethrough.

98. The method of claim 70 wherein the current is an alternating current.

99. The method of claim 98 wherein the current is applied to the localized region of the body tissue for a time period within the range of approximately 2 minutes to greater than 72 hours.

100. The method of claim 99 wherein the time period is within the range of approximately 12-72 hours.

101. The method of claim 98 wherein the current is applied at a voltage level within the range of about 1-75 V.

102. The method of claim 101 wherein the voltage level is within the range of about 1-45 V.

103. The method of claim 98 which further comprises applying a direct current prepulse prior to step (a).

104. The method of claim 98 which further comprises superimposing a direct current over the alternating current during step (a).

105. The method of claim 70 wherein the current is a direct current.

106. The method of claim 105 wherein the current is applied to the localized region of the body tissue for a time period within the range of approximately 2 minutes to greater than 72 hours.

107. The method of claim 106 wherein the time period is within the range of approximately 12-72 hours.

108. The method of claim 105 wherein the current is applied at a level within the range of about 0.01-0.5 mA/cm².

109. The method of claim 108 wherein the current is applied at a level within the range of about 0.1-0.5 mA/cm².

110. The method of claim 70 wherein the polyelectrolyte is applied to the localized region of body tissue prior to application of the current.

111. The method of claim 70 wherein the polyelectrolyte is applied to the localized region of body tissue during application of the current.

112. The method of claim 70 wherein the polyelectrolyte is applied to the localized region of body tissue both prior to and during application of the current.

113. The method of claim 70 wherein the body tissue is skin.

114. The method of claim 70 wherein the body tissues is ocular tissue.

115. The method of claim 114 wherein the ocular tissue is selected from the group consisting of conjunctiva, sclera and cornea.

116. The method of claim 70 wherein the eye tissue is mucosal tissue.

117. The method of claim 70 wherein the localized region of body tissue has an area within the range of about 0.1-100 cm².

118. The method of claim 70 which provides for at least a 20% reduction in lag-time compared to the lag-time in the absence of the polyelectrolyte.

119. The method of claim 118 which provides for at least a 40% reduction in lag-time compared to the lag-time in the absence of the polyelectrolyte.

120. The method of claim 119 which provides for at least a 60% reduction in lag-time compared to the lag-time in the absence of the polyelectrolyte.

121. The method of claim 70 which further provides for at least a 50% enhanced flux of the compound of interest compared to the flux in the absence of the polyelectrolyte.

122. The method of claim 121 which further provides for at least a 100% enhanced flux of the compound of interest compared to the flux in the absence of the polyelectrolyte.

123. The method of claim 122 which further provides for at least a 200% enhanced flux of the compound of interest compared to the flux in the absence of the polyelectrolyte.

124. The method of claim 70 wherein the compound of interest is a charged species.

125. The method of claim 70 wherein the compound of interest is an uncharged species.

126. The method of claim 70 wherein the compound of interest is an analyte extracted from within the patient's body, such that analyte is transported from beneath the localized region to the exterior of the body.

127. The method of claim 126 wherein the analyte is selected from the group consisting of glucose, galactose, lactic acid, pyruvic acid, and amino acids.

128. The method of claim 127 wherein the amino acid is selected from the group consisting of phenylalanine and tyrosine.

129. The method of claim 126 wherein the analyte is selected from the group consisting of diseases state markers, pharmacologically active agents, substances of abuse, electrolytes, minerals, hormones, amino acids, peptides, metal ions, nucleic acids, genes, enzymes, toxic agents, metabolites, conjugates, prodrugs, analogs and derivatives thereof.

130. The method of claim 126 wherein the analyte is selected from the group consisting of monosaccharides, disaccharides, oligosaccharides, organic acids, alcohols, fatty acids, cholesterol and cholesterol-based compounds, amino acids, zinc, iron, copper, magnesium, potassium, and metabolites, conjugates, prodrugs, analogs and derivatives thereof.

131. The method of claim 126 wherein the analyte is a pharmacologically active agent that has been administered to the patient.

132. The method of claim 131 wherein the pharmacologically active agent is selected from the group consisting of β-agonists; analeptic agents; analgesic agents; anesthetic agents; anti-angiogenic agents; anti-arthritic agents; anti-asthmatic agents; antibiotics; anticancer agents; anticholinergic agents; anticoagulant agents; anticonvulsant agents; antidepressant agents; antidiabetic agents; antidiarrheal agents; anti-emetic agents; anti-epileptic agents; antihelminthic agents; antihistamines; antihyperlipidemic agents; antihypertensive agents; anti-infective agents; anti-inflammatory agents; antimetabolites; antimigraine agents; antiparkinsonism drugs; antipruritic agents; antipsychotic agents; antipyretic agents; antispasmodic agents; antitubercular agents; anti-ulcer agents; antiviral agents; anxiolytic agents; appetite suppressants; attention deficit disorder and attention deficit hyperactivity disorder drugs; cardiovascular agents; central nervous system stimulants; cytotoxic drugs; diuretics; genetic materials; hormonolytics; hypnotics; hypoglycemic agents; immunosuppressive agents; muscle relaxants; narcotic antagonists; neuroprotective agents; nicotine; nutritional agents; parasympatholytics; peptide drugs; psychostimulants; sedatives; steroids; smoking cessation agents; sympathomimetics; photoactive agents; tocolytic agents; tranquilizers; vasodilators; and active metabolites thereof.

133. The method of claim 132 wherein at least two analytes are extracted concurrently.

134. The method of claim 70 wherein the compound of interest is a pharmacologically active agent to be delivered into the patient's body.

135. The method of claim 134 wherein the pharmacologically active agent is selected from the group consisting of β-agonists; analeptic agents; analgesic agents; anesthetic agents; anti-angiogenic agents; anti-arthritic agents; anti-asthmatic agents; antibiotics; anticancer agents; anticholinergic agents; anticoagulant agents; anticonvulsant agents; antidepressant agents; antidiabetic agents; antidiarrheal agents; anti-emetic agents; anti-epileptic agents; antihelminthic agents; antihistamines; antihyperlipidemic agents; antihypertensive agents; anti-infective agents; anti-inflammatory agents; antimetabolites; antimigraine agents; antiparkinsonism drugs; antipruritic agents; antipsychotic agents; antipyretic agents; antispasmodic agents; antitubercular agents; anti-ulcer agents; antiviral agents; anxiolytic agents; appetite suppressants; attention deficit disorder and attention deficit hyperactivity disorder drugs; cardiovascular agents; central nervous system stimulants; cytotoxic drugs; diuretics; genetic materials; hormonolytics; hypnotics; hypoglycemic agents; immunosuppressive agents; muscle relaxants; narcotic antagonists; neuroprotective agents; nicotine; nutritional agents; parasympatholytics; peptide drugs; psychostimulants; sedatives; steroids; smoking cessation agents; sympathomimetics; photoactive agents; tocolytic agents; tranquilizers; vasodilators; and active metabolites thereof.

136. The method of claim 134 wherein at least two pharmacologically active agents are administered simultaneously.
